(19) United States
(12) Patent Application Publication
(10) Pub. No.: US 2009/0298742 A1
(43) Pub. Date: Dec. 3, 2009

(54) PROCESS FOR MANUFACTURING LACTOSE

(75) Inventors: Trevor Charles Roche, Hertfordshire (GB); Marian Wladyslaw Wood-Kaczmar, Hertfordshire (GB); Xiang Tai, Jurong (SG); Michiel Van Oort, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GLAXO GROUP LIMITED, GREENFORD MIDDLESEX (GB)

(21) Appl. No.: 12/375,703

(22) PCT Filed: Aug. 8, 2007
(86) PCT No.: PCT/US07/75432

§ 371 Date: Jan. 30, 2009

(30) Foreign Application Priority Data
Aug. 9, 2006 (US) 60821871

(51) Int. Cl. A61K 31/58; A61K 38/02; A61K 31/56; A61K 31/573; A61K 31/46; A61K 31/47; A61K 31/18; A61K 31/135; A61K 47/36; C13K 5/00; C08B 37/00

(52) U.S. Cl. 514/2; 514/171; 514/172; 514/174; 514/178; 514/180; 514/291; 514/311; 514/603; 514/653; 514/777; 536/123.13; 536/124; 536/127

(57) ABSTRACT

A process for forming crystalline lactose suitable for use in a pharmaceutical formulation comprises subjecting a solution comprising a plurality of nanosized lactose particles to conditions sufficient to cause crystallization to occur on the nanosized lactose particles such that a plurality of lactose particles are formed therefrom having a median diameter ranging from about 4 μm to about 20 μm.

### FIELD OF INVENTION

The invention generally relates to processes for producing lactose particles.

### BACKGROUND OF THE INVENTION

In the field of inhalation therapy, it is generally desirable to employ therapeutic molecules having a particle size (i.e., diameter) in the range of 1 to 5 μm. Carrier molecules or excipients, such as lactose, for inhaled therapeutic preparations often exhibit a significantly larger diameter (e.g., 100 to 150 μm) so that they typically do not penetrate into the upper respiratory tract to the same degree as the active ingredient. However, in many instances, it is desired to use a smaller particle size for the lactose or a lactose blend having a defined ratio of coarse and fine lactose.

The lactose particle size and distribution may also, in many instances, significantly influence pharmaceutical and biological properties, such as, for example, flow properties, cohensiveness, or bioavailablity.

It is believed that one particular drawback associated with conventional means of producing pharmaceutical grade lactose relates to undesirable variations in particle size, morphology and distribution. Such production methods may be particularly problematic in that they often lead to excessive and undesirable variations in the fine particle mass (“FPMass”) of pharmaceutical formulations employing such lactose. FPMass is the weight of medicament within a given dose that reaches the desired size airways to be effective.

It would be desirable to employ a process capable of producing lactose having a more consistent particle size distribution.

### SUMMARY OF THE INVENTION

In one aspect, the invention provides a process for forming crystalline lactose having a specified median diameter. The process comprises subjecting a solution comprising a plurality of nanosized lactose particles to conditions sufficient to cause crystallization to occur on the nanosized lactose particles such that a plurality of lactose particles are formed therefrom.

These and other aspects are provided by the present invention.

### BRIEF DESCRIPTION OF THE DRAWINGS

FIG. 1 is a photograph of an apparatus used for drying lactose produced in accordance with the invention.

FIGS. 2 and 3 are SEM images of fine lactose obtained by milling and classification of pharmaceutical grade lactose (Friesland Foods Domo, Netherlands) (“conv”) and lactose produced in accordance with the invention described herein, respectively.

FIG. 4 illustrates the particle size distribution of conventional fine lactose and lactose produced in accordance with this invention and measured according to Sympatec.

FIG. 4 illustrates various particle size distributions measured according to Malvern.

FIG. 5 illustrates comparisons of particle sizes for various lactose-containing blends measured according to Malvern.

FIG. 6 illustrates comparisons of particle sizes for various lactose-containing blends measured according to Malvern.

FIG. 7 illustrates comparisons of particle sizes for various lactose-containing blends measured according to Malvern.

FIG. 8 illustrates comparisons of particle sizes for various lactose-containing blends measured according to Malvern.

FIG. 9 illustrates comparisons of particle sizes for various lactose-containing blends measured according to Sympatec.

FIG. 10 illustrates comparisons of particle sizes for various lactose-containing blends measured according to Sympatec.

FIG. 11 illustrates comparisons of particle sizes for various lactose-containing blends measured according to Sympatec.

FIG. 12 is an SEM photograph of a blend using conventional lactose.

FIG. 13 is an SEM photograph of a blend using DCL (“directly crystallized lactose”).

FIG. 14 is an SEM photograph of a blend using conventional lactose.

FIG. 15 is an SEM photograph of a blend using DCL (“directly crystallized lactose”).

FIG. 16 illustrates the compaction compressibility of various lactose-containing blends.

FIG. 17 illustrates the fine particle mass (% emitted dose) for various lactose-containing blends.

FIG. 18 illustrates the fine particle mass (% emitted dose) for various lactose-containing blends.

FIG. 19 illustrates the fine particle mass (% emitted dose) for various lactose-containing blends.

FIG. 20 illustrates the fine particle mass (% emitted dose) for various blends.

FIG. 21 illustrates Cascade impaction (CI) data for various lactose-containing blends.

FIG. 22 illustrates total impurities data for various lactose-containing blends.

FIG. 23 illustrates impurity profile data for various lactose-containing blends.

FIG. 24 illustrates assay data for various lactose-containing blends.

### DETAILED DESCRIPTION OF THE INVENTION

The invention will now be described with respect to the embodiments set forth herein including those alluded to in the drawings. It should be appreciated that these embodiments are set forth to illustrate the invention, and that the invention is not limited to these embodiments. Such embodiments mayor may not be practiced mutually exclusive of each other.

All publications, patents, and patent applications cited herein, whether supra or infra, are hereby incorporated herein by reference to their entirety to the same extent as if each publication, patent, or patent application was specifically and individually indicated to be incorporated by reference.

It must be noted that, as used in the specification and appended claims, the singular forms “a”, “an”, “the” and “one” include plural referents unless the content clearly dictates otherwise.

The terms “X50” as used herein refers to the median diameter (μm) as measured on a volume basis by a laser diffraction particle sizing system, i.e. 50% by volume of the particles are smaller than this diameter and 50% are larger. The term “X90” refers to the median diameter (μm) measured on a volume basis wherein 90% of the particles are smaller than this diameter and 10% are larger. The term “X10” refers to the median diameter (μm) measured on a volume basis wherein 10% of the particles are smaller than this diameter and 90% are larger. Measuring systems include, as an example, Sympatec HELOS system H0933 or Malvern Mastersizer 2000.

In accordance with the present invention, the term “lactose” as used herein is to be broadly construed. As an example, lactose is intended to encompass physical, crystalline, amorphous and polymorphic forms of lactose, including, but not limited to, the stereoisomers α-lactose monohydrate and β-anhydrous lactose, as well as α-anhydrous lactose. Combinations of the above may be used. Lactose (i.e., milk sugar) is preferably obtained from cheese-whey, which can be manufactured in different forms depending on the process employed. In one embodiment, the plurality of lactose particles comprise α-lactose monohydrate. In one embodiment, the plurality of lactose particles consist essentially of α-lactose monohydrate. In one embodiment, the plurality of lactose particles consist of α-lactose monohydrate. In one embodiment, the α-lactose monohydrate may have an anomeric purity of at least ninety-six (96) percent. The term “fine lactose” as used herein is to be interpreted as lactose with a median diameter (“X50”) of approximately 5 to 20 micrometers. As used herein, the term “particle” is to be broadly interpreted to encompass those of various shapes, sizes, and/or textures which can include those that may have varying degrees of irregularities, and/or disuniformities, or which my possess regular and/or uniform properties. As used herein “seed particles” is to be broadly construed to encompass lactose particles, as individually described herein, employed to initiate crystallization.

The lactose employed (i.e., “seed particles”) in the process of the invention may have various size distributions. For the purposes of this invention, the lactose seed particles are nanosized. The nanosized seed particles may have a X50 ranging from a lower end of 0.1, 0.2, 0.3, 0.4, or 0.5 μm about to a higher end of about 0.6, 0.7, 0.8, 0.9 or 1.0 μm. The nanosized lactose particles may be, for example, nanomilled lactose.

The seed particles that comprise a plurality of nanosized lactose particles may be in various solutions, any of which may be referred to as a seed suspension (“seed suspension”). For example, in one embodiment, the seed supension is a slurry of nanosized lactose seed particles in a water miscible organic solvent, any of which may be referred to as a seed slurry (“seed slurry”). In a second embodiment, the seed suspension is a slurry of nanomilled lactose particles of a size range between 0.1 and 1.0 μm. The term “miscible” as used herein is to be broadly construed to encompass both partially miscible and totally miscible solvents. The term “totally miscible” as used herein is defined as capable of mixing in any ratio without a separation of phases. The term “partially miscible” as used herein is defined as not capable of mixing in all ratios without a separation of phases. In various embodiments, the water miscible organic solvent may be selected from acetone, methanol, ethanol, tetrahydrofuran, iso-propanol and n-propanol or mixtures thereof. In one embodiment, the water miscible organic solvent is acetone.

In one embodiment of the invention, the seed suspension comprising a plurality of nanosized lactose particles may be added to a second solution prior to subjecting to conditions sufficient to cause crystallization to occur on the nanosized lactose particles. In one embodiment, the second solution may be a supersaturated lactose solution. As used herein, the term “supersaturated” refers to a condition in which the solvent is holding more solute than is stable at a given temperature. Supersaturation may be defined as the excess concentration of solute over the saturation concentration at a given temperature.

In one embodiment of the invention the second solution comprises a base. For example, the base may be NaOH, KOH, LiOH, or NaHCO₃. For example, in one embodiment, the second solution may contain 0.5 M NaOH. The 0.5 M NaOH may be 0.5, 1.0 or 2.0% solution volume of the second solution prior to the addition of seed material.

In one embodiment of the invention, the base may be added to the second solution prior to the addition of the plurality of the nanosized lactose particles and prior to subjecting the solution comprising a plurality of nanosized lactose particles to condition sufficient to cause crystallization For example, the base may be NaOH, KOH, LiOH, or NaHCO₃. For example, in one embodiment, the base may be 0.5 M NaOH.

In one embodiment, the second solution comprises a water miscible anti-solvent. For example, the anti-solvent may be acetone, methanol, ethanol, iso-propanol, n-propanol, tetrahydrofuran or mixtures thereof. In one embodiment, the anti-solvent is added to the second solution prior to seeding with a plurality of nanosized lactose particles. Additionally, in one embodiment, the second solution containing an anti-solvent may be 25, 30, 35, 40, or 45% volume anti-solvent/volume solution prior to seeding.

Moreover, in one embodiment, the second solution may contain a water miscible anti-solvent and a base.

This invention provides a process for forming crystalline lactose having a specified median diameter. The process comprises subjecting a solution comprising a plurality of nanosized lactose particles to conditions sufficient to cause crystallization to occur on the nanosized lactose particles such that a plurality of lactose particles are formed therefrom.

The step of subjecting a solution comprising a plurality of nanosized lactose particles to conditions sufficient to cause crystallization may occur under various conditions. For example, in one embodiment, such a step may occur such that the solution is linearly cooled at a rate ranging from a lower end of about −0.1, −0.2, −0.3, −0.4, −0.5° C./min to a higher end of about −1, −2, −3, −4, −5° C./min. In another embodiment, such a step may occur such that the solution is cooled at a rate of −0.6° C./min. In a third embodiment, such a step may occur such that the solution is cooled by an inverse cooling profile. In one example, not intended to be bound theory, the inverse cooling may follow an inverse cooling curve described by the equation T(t)=Tᵢ−(Tᵢ−T_{f})(t/t_{f})³, where T(t)=temperature at time t, Tᵢ32 initial temperature, T_{f}=final temperature and t_{f}=batch time. In a forth embodiment, such a step may occur such that the solution is step cooled. The term “step cooled” as used herein is defined as a cooling profile in which the solution is slowly cooled at first then cooled more rapidly as crystallization proceeds. The cooling profile may be approximated by a series of linear cooling profiles of gradually increasing cooling rate (eg any curve may be approximated as a series of interconnected straight lines). For example, a seeded solution may be cooled from 50° C. to 35° C. at −0.21° C./min followed by cooling at −0.57° C./min till 20° C.

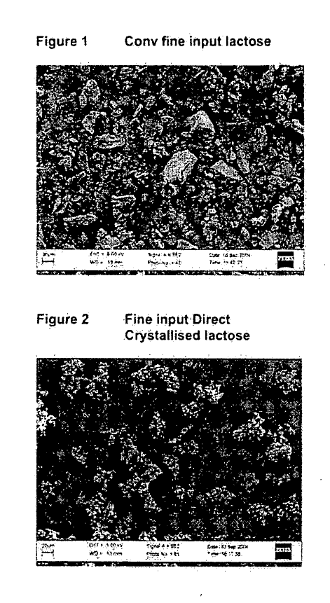

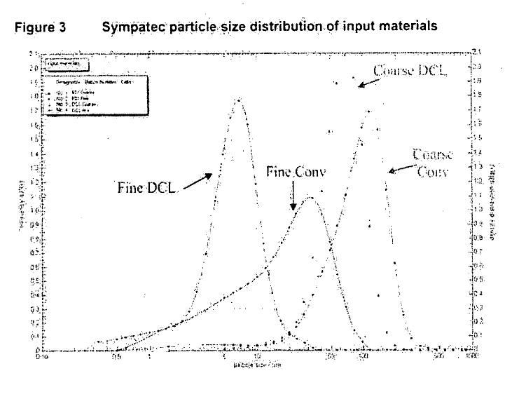

The processes of the invention may include further optional features. For example, the resulting crystallized lactose particles (“lactose slurry”) may be optionally subjected to isolation procedures. The isolated crystallized lactose particles may be optionally subjected to drying procedures. In one embodiment, the crystallized lactose particles may be filtered followed by washing with one (1) excess cake volume of 20% acetone/water, one (1) excess cake volume of 40% acetone/water followed by twice washing with one (1) excess cake volume of 100% acetone. The lactose may then be dried overnight at 40° C. in a vacuum oven. In a second embodiment, the lactose slurry may be filtered followed by washing with one (1) excess volume of 40% acetone/water solution followed by washing twice with one (1) excess cake volume of 100% acetone. The lactose may then be dried overnight at 40° C. in a vacuum oven. In an additional embodiment, the crystallized lactose particles may be dried using a contact dryer, for example, a Siemens Contact Dryer as illustrated in FIG. 1. In another embodiment, the crystallized lactose particles may be dried by centrifugation, for example, using a 5.0 μm filter with a GeneVac Ez-2 centrifuge (GeneVac Inc., Valley Cottage, N.Y.). In another example, a 10.0 μm filter may be used with a GeneVac Ez-2 centrifuge. In addition to the above, it is appreciated that other conditions known in the art may be employed.

In conjunction with the process of the invention, other procedures known in the art can be employed which are often associated with crystallization processes. Examples of such procedures include, without limitation, cleaning and sanitization, vessel pre-wash, and inter-batch cleaning. Many structural configurations may be used. For example, the process of the invention may occur in a commercial vessel. In one embodiment, for example, the process may occur in a De Dietrich Process Systems vessel, 1600 litre capacity (De Dietrich Process Systems, Inc., Union, N.J.).

The dried crystallized lactose particles produced in accordance with this invention comprise a plurality of lactose particles having a specified median diameter. The dried crystallized lactose particles may have a X50 ranging from a lower end of about 4, 5, 6, or 7 μm to higher end of about 10, 15, or 20 μm. In one embodiment, one range of median diameters would be about 4 μm to about 20 μm. In another embodiment, a range of median diameters would be about 4 μm to about 15 μm. In a third embodiment, a range of median diameters would be about 4 μm to about 10 μm. In a fourth embodiment, a range of median diameters would be about 4 μm to about 6 μm. In a fifth embodiment, a range of median diameters would be about 5 μm to about 8 μm.

The dried crystallized lactose particles produced in accordance with the described invention may be further combined with a second plurality of lactose particles having a X50 from a lower end of about 40, 50 or 60 μm to a higher end of about 70, 80, 90, or 100 μm (said second plurality of lactose particles may be referred to as “coarse lactose particles”), producing a blend of lactose particles.

In one embodiment, the crystallized lactose particles produced in accordance with the invention may be combined with at least one medicament to form a pharmaceutical formulation.

In one embodiment, a blend of lactose particles comprising dried crystallized lactose particles produced in accordance with the described invention and a second plurality of lactose particles having a X50 from a lower end of about 40, 50 or 60 μm to a higher end of about 70, 80, 90, or 100 μm may be combined with at least one medicament to form a pharmaceutical formulation.

In other aspects, the invention may encompass pharmaceutical formulations formed by the processes, as well as inhalation devices including such formulations. For example, the pharmaceutical formulation may be a dry powder pharmaceutical formulation suitable for inhalation. Medicaments, for the purposes of the invention, include a variety of pharmaceutically active ingredients, such as, for example, those which are useful in inhalation therapy. In general, the term “medicament” is to be broadly construed and include, without limitation, actives, drugs and bioactive agents, as well as biopharmaceuticals. Various embodiments may include medicament present in micronized form. Appropriate medicaments may thus be selected from, for example, analgesics, (e.g., codeine, dihydromorphine, ergotamine, fentanyl or morphine); anginal preparations, (e.g., diltiazem); anti-allergics, (e.g., cromoglicate, ketotifen or nedocromil); antiinfectives (e.g., cephalosporins, penicillins, streptomycin, sulphonamides, tetracyclines and pentamidine); antihistamines, (e.g., methapyrilene); anti-inflammatories, (e.g., anti-inflammatory steroids, beclomethasone (e.g. beclomethasone dipropionate), fluticasone (e.g. fluticasone propionate), flunisolide, budesonide, rofleponide, mometasone (e.g. mometasone furoate), ciclesonide, triamcinolone (e.g. triamcinolon acetonide), 6α,9α-difluoro-11β-hydroxy-16α-methyl-3-oxo-17α-propionyloxy-androsta-1,4-diene-17β-carbothioic acid S-(2-oxo-tetrahydrofuran-3-yl)ester), (6α,11β,16α,17α)-6,9-difluoro-17-{[(fluoromethyl)thio]carbonyl}-11-hydroxy-16-methyl-3-oxoandrosta-1,4-dien-17-yl 2-furoate, and (6α,11β,16α,17α)-6,9-difluoro-17-{[(fluoromethyl)thio]carbonyl}-11-hydroxy-16-methyl-3-oxoandrosta-1,4-dien-17-yl 4-methyl-1,3-thiazole-5-carboxylate); antitussives, (e.g., noscapine); bronchodilators, (e.g., albuterol (e.g. as sulphate), salbutamol (e.g. as the free base or the sulphate salt), salmeterol (e.g. as xinafoate), ephedrine, adrenaline, fenoterol (e.g as hydrobromide), bitolterol, formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol, 4-hydroxy-7-[2-[[2-[[3-(2-(henylethoxy)propyl]sulfonyl]ethyl]amino]ethyl-2(3H)-benzothiazolone), 3-(4-{[6-({(2R)-2-hydroxy-2-[4-hydroxy-3-(hydroxymethyl)phenyl]ethyl}amino)hexyl]oxy}butyl)benzenesulfonamide, 3-(3-{[7-({(2R)-2-hydroxy-2-[4-hydroxy-3-(hydroxymethyl)phenyl]ethyl}amino)heptyl]oxy}propyl)benzenesulfonamide, 4-{(1R)-2-[(6-{2-[(2,6-dichlorobenzyl)oxy]ethoxy}hexyl)amino]-1-hydroxyethyl}-2-(hydroxymethyl)phenol, 2-hydroxy-5-((1R)-1-hydroxy-2-{[2-(4-{[(2R)-2-hydroxy-2-phenylethyl]amino}phenyl)ethyl]amino}ethyl)phenylformamide, and 8-hydroxy-5-{(1R)-1-hydroxy-2-[(2-{4-[(6-methoxy-1,1′-biphenyl-3-yl)amino]phenyl}ethyl)amino]ethyl}quinolin-2(1H)-one); diuretics, (e.g., amiloride); anticholinergics, (e.g., ipatropium (e.g., as bromide), tiotropium, atropine or oxitropium); hormones, (e.g., cortisone, hydrocortisone or prednisolone); xanthines, (e.g., aminophylline, choline theophyllinate, lysine theophyllinate or theophylline); therapeutic proteins and peptides, (e.g., insulin). In addition to those stated above, it will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimize the activity and/or stability of the medicament. It will be further clear to a person skilled in the art that where appropriate, the medicaments may be used in the form of a pure isomer, for example, R-salbutamol or RR-formoterol.

Particular medicaments for administration using pharmaceutical formulations in accordance with the invention include anti-allergies, bronchodilators, beta agonists (e.g., long-acting beta agonists), and anti-inflammatory steroids of use in the treatment of respiratory conditions, as defined herein, by inhalation therapy, for example, cromoglicate (e.g. as the sodium salt), salbutamol (e.g. as the free base or the sulphate salt), salmeterol (e.g. as the xinafoate salt), bitolterol, formoterol (e.g. as the fumarate salt), terbutaline (e.g. as the sulphate salt), 3-(4-{[6-({(2R)-2-hydroxy-2-[4-hydroxy-3-(hydroxymethyl)phenyl]ethyl}amino)hexyl]oxy}butyl)benzenesulfonamide, 3-(3-{[7-({(2R)-2-hydroxy-2-[4-hydroxy-3-(hydroxymethyl)phenyl]ethyl}amino)heptyl]oxy}propyl)benzenesulfonamide, 4-{(1R)-2-[(6-{2-[(2,6-dichlorobenzyl)oxy]ethoxy}hexyl)amino]-1-hydroxyethyl}-2-(hydroxymethyl)phenol, 2-hydroxy-5-((1R)-1-hydroxy-2-{[2-(4-{[(2R)-2-hydroxy-2-phenylethyl]amino}phenyl)ethyl]amino}ethyl)phenylformamide, 8-hydroxy-5-{(1R)-1-hydroxy-2-[(2-{4-[(6-methoxy-1,1′-biphenyl-3-yl)amino]phenyl}ethyl)amino]ethyl}quinolin-2(1H)-one, reproterol (e.g. as the hydrochloride salt), a beclomethasone ester (e.g. the dipropionate), a fluticasone ester (e.g. the propionate), a mometasone ester (e.g., the furoate), budesonide, dexamethasone, flunisolide, triamcinolone, tripredane, (22R)-6α,9α-difluoro-11β,21-dihydroxy-16α,17α-propylmethylenedioxy-4-pregnen-3,20-dione. Medicaments useful in erectile dysfunction treatment (e.g., PDE-V inhibitors such as vardenafil hydrochloride, along with alprostadil and sildenafil citrate) may also be employed. It should be understood that the medicaments that may be used in conjunction with the inhaler are not limited to those described herein.

Salmeterol, especially salmeterol xinafoate, salbutamol, fluticasone propionate, beclomethasone dipropionate and physiologically acceptable salts and solvates thereof are especially preferred.

It will be appreciated by those skilled in the art that the formulations according to the invention may, if desired, contain a combination of two or more medicaments. Formulations containing two active ingredients are known for the treatment and/or prophylaxis of respiratory disorders such as those described herein, for example, formoterol (e.g. as the fumarate) and budesonide, salmeterol (e.g. as the xinafoate salt) and fluticasone (e.g. as the propionate ester), salbutamol (e.g. as free base or sulphate salt) and beclomethasone (as the dipropionate ester) are preferred.

In one embodiment, a particular combination that may be employed is a combination of a beta agonist (e.g., a long-acting beta agonist) and an anti-inflammatory steroid. One embodiment encompasses a combination of salmeterol, or a salt thereof (particularly the xinafoate salt) and fluticasone propionate. The ratio of salmeterol to fluticasone propionate in the formulations according to the present invention is preferably within the range 4:1 to 1:20. The two drugs may be administered in various manners, simultaneously, sequentially, or separately, in the same or different ratios. In various embodiments, each metered dose or actuation of the inhaler will typically contain from 25 μg to 100 μg of salmeterol and from 25 μg to 500 μg of fluticasone propionate. The pharmaceutical formulation may be administered as a formulation according to various occurrences per day. In one embodiment, the pharmaceutical formulation is administered twice daily.

Embodiments of specific medicament combinations that may be used in various pharmaceutical formulations are as follows:

1) fluticasone propionate 100 μg/salmeterol 50 μg

2) fluticasone propionate 250 μg/salmeterol 50 μg

3) fluticasone propionate 500 μg/salmeterol 50 μg

In various embodiments, the pharmaceutical formulations may be present in the form of various inhalable formulations. In one embodiment, the pharmaceutical formulation is present in the form of a dry powder formulation, the formulation of such may be carried out according to known techniques. Dry powder formulations for topical delivery to the lung by inhalation may, for example, be presented in capsules and cartridges of, for example, gelatine, or blisters of, for example, laminated aluminum foil, for use in an inhaler or insufflator. Powder blend formulations generally contain a powder mix for inhalation of the compound of the invention and a suitable powder base which includes lactose and, optionally, at least one additional excipient (e.g., carrier, diluent, etc.). In various embodiments, each capsule or cartridge may generally contain between 20 μg and 10 mg of the at least one medicament. In one embodiment, the formulation may be formed into particles comprising at least one medicament, and excipient material(s), such as by co-precipitation or coating. When employed as a dry powder, packaging of the formulation may be suitable for unit dose or multi-dose delivery. In the case of multi-dose delivery, the formulation can be pre-metered (e.g., as in Diskus®, see GB 2242134/U.S. Pat. Nos. 6,032,666, 5,860,419, 5,873,360, 5,590,645, 6,378,519, 6,536,427, and 6,792,645 or Diskhaler, see GB 2178965, 2129691 and 2169265, U.S. Pat. Nos. 4,778,054, 4,811,731, 5,035,237) or metered in use (e.g. as in Turbuhaler, see EP 69715, or in the devices described in U.S. Pat. No. 6,321,747). An example of a unit-dose device is Rotahaler® (see GB 2064336). In one embodiment, the Diskus® inhalation device comprises an elongate strip formed from a base sheet having a plurality of recesses spaced along its length and a lid sheet hermetically but peelably sealed thereto to define a plurality of containers, each container having therein an inhalable formulation containing the at least one medicament, the lactose, optionally with other excipients. Preferably, the strip is sufficiently flexible to be wound into a roll. The lid sheet and base sheet will preferably have leading end portions which are not sealed to one another and at least one of the leading end portions is constructed to be attached to a winding means. Also, preferably the hermetic seal between the base and lid sheets extends over their whole width. The lid sheet may preferably be peeled from the base sheet in a longitudinal direction from a first end of the base sheet.

The pharmaceutical formulation formed by the processes of the invention may be used in the treatment of a number of respiratory disorders. Such respiratory conditions include, without limitation, diseases and conditions associated with reversible airways obstruction such as asthma, chronic obstructive pulmonary disease (e.g. chronic and wheezy bronchitis, emphysema), respiratory tract infection and upper respiratory tract disease (e.g. rhinitis, such as allergic and seasonal rhinitis). Such treatment is carried out by delivering medicament to a mammal. In will be appreciated by those skilled in the art that reference herein to “treatment” extends to prophylaxis as well as addressing established conditions. Accordingly, and in view of the above, in another aspect, the invention provides a method for the treatment of a respiratory disorder comprising the step of administering a pharmaceutical effective amount of a pharmaceutical formulation to a mammal such as, for example, a human. For the purposes of the invention, the term “pharmaceutically effective amount” is to be broadly interpreted and encompass the treatment of the disorder. In one embodiment, the administration is carried out via an inhalation device described herein. In one embodiment, the administration is carried out by nasal or oral inhalation.

The present invention also encompasses crystalline lactose particles. The crystalline lactose particles may be produced according to any of the processes disclosed herein. The crystallized lactose produced in accordance with this invention appears to have smoother surfaces and a more uniform particle size than conventional fine lactose. The lactose may be crystallized such that lactose monohydrate results. The lactose particles may be directly crystallized, i.e., be formed from a single batch. In a first embodiment, the particle size of the crystallized lactose particles produced in accordance with this invention is characterized by an X10 of approximately 1 micron and an X90 of approximately 20 microns. In a second embodiment, the particle size of the crystallized lactose particles produced in accordance with this invention is characterized by an X10 of approximately 2 microns and an X90 of approximately 15 microns. Any of the above embodiments may have a logarithmic particle distribution that is Gaussian.

The lactose produced may have a uniform, narrow particle size distribution and the individual particles may be smooth and undamaged by milling.

The flow properties of the DCL lactose formulations may appear to be

less affected by the addition of cellobiose octa-acetate (COA) than the corresponding conventional fine lactose formulations. Potentially more COA could be added to DCL lactose formulations with any or little affect on the flow properties and may not affect the filling performance.

The following examples are intended to illustrate the invention, and do not limit the scope of the invention as defined by the claims.

Table 1 sets forth solutions and methods employed in the crystallization embodiments illustrated in the Examples.

TABLE 1
Solutions/Methods

| Experimental Method | Description |
|---|---|
| Lactose Solution A | 32 g of Conventional Lactohale Grade 4 |
| | milled lactose was dissolved in 30 ml of |
| | water by heating the mixture to 90° C. |
| Seeding Method A | Using a Gilson pipette, the seed slurry* |
| | volume that had the equivalent lactose |
| | quantity needed was pipetted into the |
| | crystallising solution. |
| Seeding Method B | Using a syringe pipette, the mass of seed |
| | slurry* that had the equivalent lactose |
| | quantity needed was added into the |
| | crystallising solution. |
| Isolation Method A | The lactose slurry was filtered, followed |
| | by washing with 1 excess cake volume of |
| | 20% acetone/water, 1 excess cake |
| | volume of 40% acetone/water and |
| | repeating twice, washing with 1 excess |
| | cake volume of 100% acetone. The |
| | lactose was then dried overnight at 40° C. |
| | in a vacuum oven. |
| Isolation Method B | The lactose slurry was filtered, followed |
| | by washing with 1 excess volume of 40% |
| | acetone/water solution followed by |
| | repeating twice, washing with 1 excess |
| | cake volume of 100% acetone. The |
| | lactose was then dried overnight at 40° C. |
| | in a vacuum oven. |

The seed slurry was prepared using 0.2-0.3 micron nanomilled lactose particles. The size of the particles was measured by scanning electron microscopy. The lactose was nanomilled using a Drais Cosmo 5 bead mill (Bühler GmbH, Zweigniederlassung Mannheim, Grinding and Dispersing Technology, Grosser Stellweg 16, 68519 Viernheim, Germany) using zirconium oxide beads. 2.5 kg of micronised lactose particles was suspended in 25 L of acetone. The suspension was cycled through the mill set to a rotor speed of about 1400 rpm and a power input of about 3.4 kw. The milling was continued for about 15 hours.

“Sympatec” refers to Sympatec GmbH located at System-Partikel-Technik, Am Pulverhaus 1, D-38678 Clausthal-Zellerfeld, Germany.

### EXAMPLE 1

### Crystallization Procedure

Lactose Solution A was cooled to 50° C. and seeded with 60 mg of seed using Seeding Method A. The slurry was then cooled from 50° C. to 20° C. over ten (10) hours, following an inverse cooling curve described by the equation T(t)=Tᵢ−(Tᵢ−T_{f})(t/t_{f})³, where T(t)=temperature at time t, tᵢ=initial temperature, T_{f}=final temperature and t_{f}=batch time. The lactose was isolated using Isolation Method A. The lactose was of X50=16.62 μm.

### EXAMPLE 2

### Crystallization Procedure

Lactose Solution A was cooled to 50° C. seeded with 180 mg of seed using Seeding Method A. The slurry was then cooled to 20° C. using a linear cooling rate of −0.6° C./min. The lactose was isolated using Isolation Method A. The X50 was of 8.96 μm.

### EXAMPLE 3

### Crystallization Procedure

1% solution volume of 0.5 M NaOH was added to Lactose Solution A at 90° C. prior to cooling to 50° C. and seeding with 180 mg using Seeding Method A. The slurry was copied to 20° C. using a linear cooling rate of −0.6° C./min. The lactose was isolated using isolation Method A. The X50 was of 8.53 μm.

### EXAMPLE 4

### Crystallization Procedure

2% solution volume of 0.5 M NaOH was added to Lactose Solution A at 90° C. prior to cooling to 50° C. and seeding with 180 mg using Seeding Method A. The slurry was cooled to 20° C. using a linear cooling rate of −0.6° C./min. The lactose was isolated using Isolation Method A. The X50 was of 9.96 μm.

### EXAMPLE 5

### Crystallization Procedure

3% solution volume of 0.5 M NaOH was added to Lactose Solution A at 90° C. prior to cooling to 50° C. and seeding with 180 mg using Seeding Method A. The slurry was copied to 20° C. using a linear cooling rate of −0.6° C./min. The lactose was isolated using Isolation Method A. The X50 was of 9.88 μm.

### EXAMPLE 6

### Crystallization Procedure

4% solution volume of 0.5 M NaOH was added to Lactose Solution A at 90° C. prior to cooling to 50° C. and seeding with 180 mg using Seeding Method A. The slurry was cooled to 20° C. using a linear cooling rate of −0.6° C./min. The lactose was isolated using Isolation Method A. The X50 was of 10.31 μm.

### EXAMPLE 7

### Crystallization Procedure

25% v/v ethanol/water solution was added to Lactose Solution A at 60° C. The Solution was then seeded with 180 mg of seed. The seeded solution was linear cooled at −0.6° C./min from 60° C. to 20° C. Lactose was isolated using Isolation Method A. The X50 of the resulting lactose was 8.61 μm.

### EXAMPLE 8

### Crystallization Procedure

25% v/v acetone/water solution was added to Lactose Solution A at 55° C. The Solution was then seeded with 180 mg of seed. The seeded solution was linear cooled at −0.6° C./min from 55° C. to 20° C. Lactose was isolated using Isolation Method A. The X50 of the resulting lactose was 6.79 μm.

### EXAMPLE 9

### Crystallization Procedure

45% v/v acetone/water solution was added to Lactose Solution A at 50° C.; the resulting solution was seeded with 500 mg of seed at 50° C. using Seeding Method B. The seeded solution was linear cooled at −0.43° C./min from 50° C. to 20° C. Lactose was isolated using Isolation Method A. The X50 of the resulting lactose was 5.63 μm.

### EXAMPLE 10

### Crystallization Procedure

45% v/v ethanol/water solution was added to Lactose Solution A at 50° C.; the resulting solution was seeded with 500 mg of seed at 50° C. using Seeding Method B. The seeded solution was linear cooled at −0.43° C./min from 50° C. to 20° C. Lactose was isolated using Isolation Method A. The X50 of the resulting lactose was 5.08 μm.

### EXAMPLE 11

### Crystallization Procedure

45% v/v ethanol/water solution was added to Lactose Solution A at 50° C.; the resulting solution was seeded with 500 mg of seed at 50° C. using Seeding Method B. The seeded solution was linear copied at −0.43° C./min from 50° C. to 20° C. Lactose was isolated using Isolation Method B. The X50 of the resulting lactose was 4.99 μm.

### EXAMPLE 12

### Crystallization Procedure

30% v/v acetone/water solution was added to Lactose Solution A with 1% 0.5 M NaOH at 50° C.; the resulting solution was seeded with 500 mg of seed at 50° C. using Seeding Method B. The seeded solution was step cooled from 50° C. to 35° C. at −0.21° C./min followed by cooling at −0.57° C./min till 20° C. Lactose was isolated using Isolation Method B. The X50 of the resulting lactose was 6.55 μm.

### EXAMPLE 13

### Crystallization Procedure

40% v/v acetone/water solution was added to Lactose Solution A at 50° C.; the resulting solution was seeded with 500 mg of seed at 50° C. using Seeding Method B. The seeded solution was step cooled from 50° C. to 35° C. at −0.21° C./min followed by cooling at −0.57° C./min till 20° C. Lactose was isolated using Isolation Method A. The X50 of the resulting lactose was 4.36 μm.

### EXAMPLE 14

### Crystallization Procedure

384 g of lactose was dissolved in 360 ml of water by heating at 90° C. 40% v/v acetone/water solution was added to the lactose solution at 50° C.; the resulting solution was seeded with 6 g of seed at 50° C. using Seeding Method B. The seeded solution was step cooled from 50° C. to 35° C. at −0.21° C./min followed by cooling at −0.57° C./min till 20° C. Lactose was isolated using Isolation Method B. The X50 of the resulting lactose was 5.81 μm.

### EXAMPLE 15

### Crystallization Procedure

40% v/v acetone/water solution was added to Lactose Solution A at 50° C.; the resulting solution was seeded with 500 mg of seed at 50° C. using Seeding Method B. The seeded solution was linear cooled at −0.43° C./min from 50° C. to 20° C. Lactose was isolated using Isolation Method B. The X50 of the resulting lactose was 6.44 μm.

### EXAMPLE 16

### Crystallization Procedure

224 g of lactose was dissolved in 210 ml of water by heating at 90° C. 40% v/v acetone/water solution was added to the lactose solution at 50° C.; the resulting solution was seeded with 3.5 g of seed at 50° C. using Seeding Method B. The seeded solution was linear cooled at −0.43° C. 7 min from 50° C. to 20° C. Lactose was isolated using Isolation Method B. The X50 of the resulting lactose was 6.13 μm.

### EXAMPLE 17

### Crystallization Procedure

384 g of lactose (Lactose New Zealand batch “A′”) was dissolved in 360 ml of water by heating at 90° C. 40% v/v acetone/water solution was added to the lactose solution at 50° C.; the resulting solution was seeded with 6 g of seed at 50° C. using Seeding Method B. The seeded solution was step cooled from 50° C. to 35° C. at −0.21° C./min followed by cooling at −0.57° C./min till 20° C. Lactose was isolated using Isolation Method B. The lactose was re-suspended in acetone, de-liquored by filtration and the wet cake dried using a Siemens custom built, laboratory contract dryer consisting of an agitated, heated vacuum chamber. The material was dried at 200 mbar, 30° C., 10 rpm agitator speed. The input wet weight was 37.67 g. The final dry weight was 24.84 grams. The solvent mass fraction was 34%. The weight loss during drying was monitored and recorded throughout. The X50 of the resulting lactose was 5.77 μm.

### EXAMPLE 18

### Conventional Lactose and Lactose Produced in Accordance with this Invention

Scanning electron micrographs of (a) conventional fine lactose (Lot “A”, Friesland Foods Domo, Netherlands) (“Conv Fine”) and (b) fine lactose produced in accordance with this invention. A small amount, less than 10 mg, of each sample was finely dispersed using a small brush, onto a carbon electrodag tab stuck onto an aluminum SEM stub. These were then coated with gold using an EMSCOPE FD500 Sputter Coating Unit (Quorum Technologies, UK). The samples were then imaged on a Philips XL120 Scanning Electron Micrograph. FIG. 1 represents an SEM of conventional input lactose and FIG. 2 represents an SEM photograph of lactose produced according to the invention.

### EXAMPLE 19

### Conventional Lactose and Lactose Produced in Accordance with this Invention

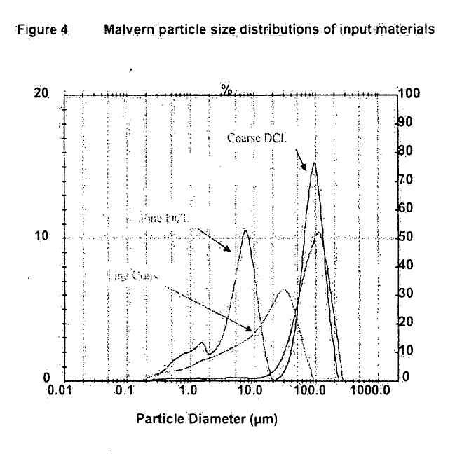

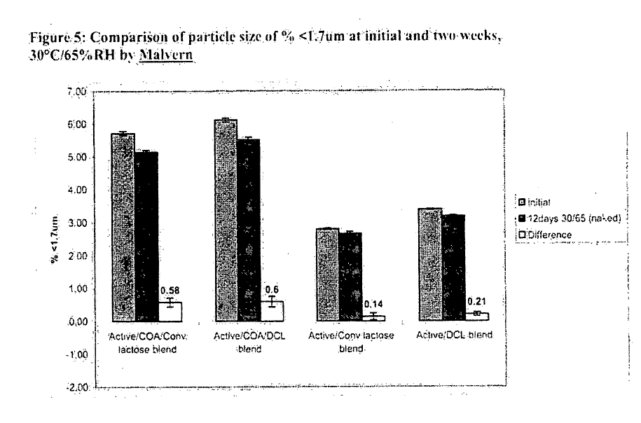

Particle size distribution (“PSD”) of conventional fine lactose (Lot “A”, Friesland Foods Domo, Netherlands) (“Fine Conv”), lactose produced in accordance with this invention (“Fine DCL”), conventional coarse lactose (Friesland Foods Domo, Netherlands) (“Course Conv”) and lactose produced according to Ser. No. 60/821,872 copending application entitled “Process for Manufacturing Lactose” filed concurrently herewith (“Course DCL”) q3*(x)=cumulative distribution. q3lg(x)=log density distribution. Particle size distributions were compared by identical particle sizing methods using a Sympatec particle sizer. For each analysis a 2±1 g sample is transferred into the funnel of the Vibri feeder using a Kartell general purpose spatula (Fisher catalogue no. SMG-410-091M, volume approximately 1.8 cm³). The sample is then dispersed by the Vibri feeder (Sympatec) and the Rodos disperser (Sympatec) before entering the Sympatec HELOS laser diffraction particle sizer, model—BF or KF. Parameters: 1.5 bar, R5 lens. FIG. 4 illustrates the various particle size distributions.

### EXAMPLE 20

### Conventional Lactose and Lactose Produced in Accordance with this Invention

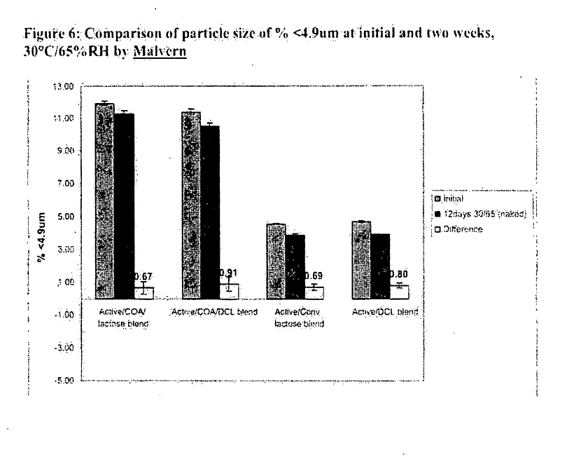

Particle size distribution (“PSD”) of conventional fine lactose (Lot “A”, Friesland Foods Domo, Netherlands) (“Fine Conv”), lactose produced in accordance with this invention (“Fine DCL”), conventional coarse lactose (“Course Conv”) and lactose produced according to Ser. No. 60/821,872 copending application entitled “Process for Manufacturing Lactose” filed concurrently herewith (“Course DCL”). Particle size distributions were compared by identical particle sizing methods using a Malvern wet dispersion method. y-axis left: volume percentage. y-axis right: cumulative volume percentage. FIG. 5 illustrates the various particle size distributions.

### EXAMPLE 21

### Conventional Lactose and Lactose Produced in Accordance with this Invention

Particle size distribution (“PSD”) of conventional fine lactose (Lot “A”, Friesland Foods Domo, Netherlands) (“conv”), lactose produced in accordance with this invention (“DCL”), conventional coarse lactose (Friesland Foods Domo, Netherlands) and lactose produced according to Ser. No. 60/821,872 copending application entitled “Process for Manufacturing Lactose” filed concurrently herewith. Particle size was measured using a Sympatec particle sizer and using a Malvern wet disperson method.

For each Sympatec analysis a 2±1 g sample is transferred into the funnel of the Vibri feeder using a Kartell general purpose spatula (Fisher catalogue no. SMG-410-091M, volume approximately 1.8 cm³). The sample is then dispersed by the Vibri feeder and the Rodos disperser before entering the Sympatec HELOS laser diffraction particle size, model—BF or KF. Parameters: 1.5 bar, R5 lens. X50=D(v, 0.5). Table 2 lists the sizing data.

TABLE 2
Summary of the data

| | Sympatec Data | | | Malvern data | | |
|---|---|---|---|---|---|---|
| Sizing of Input materials | X50 um | % <4.5 um | % <15 um | D(v, 0.5) um | % <4.9 um | % <14.2 um |
| Input Conv Coarse lactose | 93.18 | 2.41 | 4.21 | 87.20 | 4.40 | 6.20 |
| Input Conv Fine lactose | 19.42 | 15.72 | 41.38 | 14.80 | 26.20 | 49.00 |
| Input Coarse DCL | 64.41 | 0.16 | 1.75 | 88.70 | 2.61 | 2.96 |
| Input Fine DCL | 6.13 | 29.48 | 97 | 5.30 | 46.30 | 98.30 |
| Input Mic. COA | 1.44 | 99.85* | 100 | 1.69 | 98.95 | 100.00 |
| Input Mic. active | 3.00 | 86.00* | 100 | 2.15 | 96.15 | 100.00 |

% <5 micron data was reported for input COA and input micronised active, as defined by the release specifications of these materials.

### EXAMPLE 22

### Conventional Lactose and Lactose Produced in Accordance with this Invention

Particle size span of lactose produced in accordance with this invention (“DCL”), conventional fine lactose (Lot “B”, Friesland Foods Domo, Netherlands) (“Conv”), lactose produced according to Ser. No. 60/821,872 copending application entitled “Process for Manufacturing Lactose” filed concurrently herewith, and conventional coarse lactose (Lot C′, Friesland Foods Domo, Netherlands). Span=(X90−X10)/X50 from Sympatec data. Span is a measure of width of particle size distribution. X90=particle diameter corresponding to 10% of the cumulative undersize distribution by volume, μm. X10=particle diameter corresponding to 90% of the cumulative undersize distribution by volume, μm. X50=particle diameter corresponding to 50% of the cumulative undersize distribution by volume, μm. Table 3 lists these values For each Sympatec analysis a 2±1 g sample is transferred into the funnel of the Vibri feeder using a Kartell general purpose spatula (Fisher catalogue no. SMG-410-091M, volume approximately 1.8 cm³). The sample is then dispersed by the Vibri feeder and the Rodos disperser before entering the Sympatec HELOS laser diffraction particle sizer, model—BF or KF (Sympatec). Parameters: 1.5 bar, R5 lens.

TABLE 3
Calculation of Span.

| Material | X10 | X50 | X90 | Span |
|---|---|---|---|---|
| coarse DCL | 32.95 | 64.41 | 108.48 | 1.17 |
| coarse conv | 32.88 | 93.18 | 171.54 | 1.49 |
| fine DCL | 2.26 | 6.13 | 11.27 | 1.47 |
| fine conv | 3.04 | 19.42 | 49.56 | 2.40 |

Span = (D90 − D10)/D50 from Sympatec data

### EXAMPLE 23

### Blends of Conventional Lactose and Lactose Produced in Accordance with this Invention

Particle size of in-process and finished blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide (“active”) with and without cellobiose octa-acetate (“COA”) and lactose produced according to this invention combined With lactose produced according to Ser. No. 60/821,872 copending application entitled “Process for Manufacturing Lactose” filed concurrently herewith (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA. Blends were sized by both the Sympatec (dry disperson) and Malvern (wet disperson) methods. In-process blends contain only the combined lactose. Finished blends contain 3-(4-{[6-({(2R)-2-hydroxy-2-[4-hydroxy-3-(hydroxymethyl)phenyl]ethyl}amino)hexyl]oxy}butyl)benzenesulfonamide. Table 4 sets forth various particle sizes.

These blends were prepared according to Table 5. The DC lactose/COA blend is prepared as follows: (1) Coarse DCL is sieved through a 710 μm sieved; (2) Approximately 857 g of coarse DCL is added to a TRV8 blender [GEA Aeromatic Fielder Ltd, GEA Process Engineering Ltd., United Kingdom]; (3) Approximately 119 g of fine DCL is added to the top of coarse DC lactose in the blender; (4) Approximately 857 g of coarse DCL is added on top of the fine DC lactose; (5) The lactose is blended for 1 minute at 575 rpm; (6) 29 g of the DC lactose mixture is removed; (7) The remaining DC lactose mixture is blended for 1 min at 575 rpm; (8) Approximately 229 g of the DC lactose mixture is removed; (9) Approximately 175 g of COA is sandwiched between the DC lactose remaining in the blender; (10) The DC lactose and COA mixture is blended for 10 mins at 570 rpm; (11) 11 g of the DC lactose/COA mixture is removed; (12) Approximately 2.31 g of 3-(4-{[6-({(2R)-2-hydroxy-2-[4-hydroxy-3-(hydroxymethyl)phenyl]ethyl}amino)hexyl]oxy}butyl)benzenesulfonamide (“active”) is mixed with approximately 25 g of the DC lactose/COA mixture in a stainless steel bowl with stainless steel spatula. This DC lactose/COA/drug substance mixture is sandwiched between the DC lactose/COA mixture remaining in blender. The bowl is dry rinsed 3 times with the DC lactose/COA mix; (13) This final DC lactose/COA/drug substance mixture is blended 570 rpm for 10 mins.

The CL/COA blend is prepared as described for the DC lactose/COA blend as described in previously paragraph except that in step (2) Approximately 872.5 g of coarse CL is used; (3) Approximately 88 g fine CL is used; and (4) Approximately 82.5 g of coarse CL is used, for a total of 1745 g coarse CL.

The DC lactose binary blend is prepared using 200 g of the lactose pre-mix from the first stage of the DC lactose/COA blend from step 8 above. 50 g of this DC/COA blend was placed in a QMM blender with 1 L bowl (Donsmark Process Technology, Denmark). Approximately 0.264 g of drug substance was mixed with approx 5 g of the DC lactose/COA blend using a stainless steel container and spatula before being added to top of blender. A further 50 g of the DC lactose/COA blend is added to the top of the blender. This DC lactose/COA/drug mixture is then blended at 750 rpm for 10 mins. The remaining DC lactose/COA blend is added to the top of blender and is blended for 9 mins at 750 rpm. The blend is then removed and sievee through a 500 μm sieve. The blend is returned to the blender and further blended for 1 min at 750 rpm.

The CL binary blend is prepared as described for the DC lactose binary blend but using lactose pre-mix from the BDI/COA rather than the DCL/COA pre-mix.

The relative humidity of the room during blending was between 48 and 60 percent. The temperature of the room was between 18 and 20° C.

For each Sympatec analysis a 2±1 g sample is transferred into the funnel of the Vibri feeder using a Kartell general purpose spatula (Fisher catalogue no. SMG-410-091M, volume approximately 1.8 cm³). The sample is then dispersed by the Vibri feeder and the Rodos disperser before entering the Sympatec HELOS laser diffraction particle sizer, model—BF or KF. Parameters: 1 bar, R4 lens.

Blend uniformity is believed to be observed as shown in Table 6. % w/w is the given mass of a component in the lactose blend. For example 0.1% w/w 3-(4-{[6-({(2R)-2-hydroxy-2-[4-hydroxy-3-(hydroxymethyl)phenyl]ethyl}amino)hexyl]oxy}butyl)benzenesulfonamide and with 10% COA would be by mass 0.1% 3-(4-{[6-({(2R)-2-hydroxy-2-[4-hydroxy-3-(hydroxymethyl)phenyl]ethyl}amino)hexyl]oxy}butyl)benzenesulfonamide, 10% COA, 89.9% lactose.

TABLE 4
Particle size of in-process and final blends

| | Sympatec | | | | Malvern | | | |
|---|---|---|---|---|---|---|---|---|
| Sample details | X50 um | % <1.8 um | % <4.5 um | % <15 um | D(v, 0.5) um | % <1.7 um | % <4.9 um | % <14.2 um |
| In-Process Blends | | | | | | | | |
| conv Lactose blend | 97.90 | 1.39 | 2.91 | 5.44 | 87.54 | 2.86 | 4.36 | 7.03 |
| (2 mins) | | | | | | | | |
| DCL blend (2 mins) | 65.67 | 0.18 | 2.1 | 7.69 | 74.43 | 3.16 | 4.01 | 5.66 |
| Conv Lactose/COA | 72.45 | 14.93 | 25.15 | 27.04 | 83.67 | 5.42 | 11.86 | 14.86 |
| blend | | | | | | | | |
| DCL/COA blend | 55.96 | 11.98 | 21.72 | 26.76 | 70.15 | 5.38 | 10.30 | 14.21 |
| Finished Blends | | | | | | | | |
| conv Lactose/active | 91.05 | 1.89 | 3.68 | 6.25 | 88.93 | 2.81 | 4.57 | 7.13 |
| blend | | | | | | | | |
| DCL/active blend | 62.15 | 0.82 | 4.04 | 9.98 | 72.34 | 3.38 | 4.71 | 7.63 |
| conv | 71.30 | 14.87 | 24.67 | 26.62 | 82.25 | 5.76 | 11.94 | 14.92 |
| Lactose/active/COA | | | | | | | | |
| blend | | | | | | | | |
| DCL/active/COA blend | 54.75 | 11.97 | 21.77 | 26.09 | 67.99 | 6.17 | 11.41 | 15.39 |

TABLE 5
List of active formulation manufactured and analysed

| | | | | | | | | RH/temp of |
|---|---|---|---|---|---|---|---|---|
| | | | | | | | | the blend |
| | | | | Blender | | | | immediately |
| | | | | speed | Pre- | | | post- |
| Components | Scale | | Blender | (rpm) | mix | Stage 1 | Stage 2 | blending |
| 0.1% w/w active, | 200 | g | QMM | 750 | 2 min | 10 min | 10 min | Not recorded |
| conv lactose | | | | | | | | |
| (6% fines | | | | | | | | |
| <4.5 μm) | | | | | | | | |
| 0.1% w/w active + | 1.75 | kg | TRV8 | 570 | 2 min | 10 min | 10 min | 65.2% & 21.0° C. |
| 10% COA, conv | | | | | | | | |
| lactose (6% | | | | | | | | |
| fines <4.5 μm) | | | | | | | | |
| 0.1% w/w active, | 200 | g | QMM | 750 | 2 min | 10 min | 10 min | 46.5% & 23.2° C. |
| DCL lactose (6% | | | | | | | | |
| fines <4.5 μm) | | | | | | | | |
| 0.1% w/w active + | 1.75 | kg | TRV8 | 570 | 2 min | 10 min | 10 min | 55.8% & 19.7° C. |
| 10% COA, DCL | | | | | | | | |
| lactose (6% | | | | | | | | |
| fines <4.5 μm) | | | | | | | | |

Premix * = blending coarse and fine lactose (the premix was prepared in bulk for both QMM and TRV8 blends).

TABLE 6
Blend Uniformity Data

| | Mean | Relative | Mean | Relative |
|---|---|---|---|---|
| | active | Standard | COA | Standard |
| | content | Deviation | content | Deviation |
| Components | (% w/w) | (%) | (% w/w) | (%) |
| 0.1% w/w active + | 0.0977 | 0.69 | n/a | n/a |
| conv lactose | | | | |
| 0.1% w/w active + | 0.1015 | 1.42 | 9.499 | 3.19 |
| 10% COA + | | | | |
| conv lactose | | | | |
| 0.1% w/w active + | 0.0971 | 0.38 | n/a | n/a |
| DC lactose | | | | |
| 0.1% w/w active + | 0.0993 | 2.86 | 9.532 | 4.13 |
| 10% COA + | | | | |
| DC lactose | | | | |

(n = 10)

### EXAMPLE 24

### Blends of Conventional Lactose and Lactose Produced in Accordance with this Invention

Particle size of blends of conventional fine lactose (Lot “A”, Friesland Foods Domo, Netherlands) and conventional coarse lactose (Lot “C”, Friesland Foods Domo, Netherlands) (“CL blend”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and lactose produced according to this invention combined with lactose produced according to Ser. No. 60/821,872 copending application entitled “Process for Manufacturing Lactose” filed concurrently herewith. (“DC lactose”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA. Blends were sized by both the Sympatec (dry disperson) and Malvern (wet disperson) methods. Particle size was measured initially and after two weeks exposure at 30° C./65% relative humidity. Table 7 [Table 8, Ware TM] For each Sympatec analysis a 2±1 g sample is transferred into the funnel of the Vibri feeder using a Kartell general purpose spatula (Fisher catalogue no. SMG-410-091M, volume approximately 1.8 cm³). The sample is then dispersed by the Vibri feeder and the Rodos disperser before entering the Sympatec HELOS laser diffraction particle sizer, model—BF or KF. Parameters: 1 bar, R4 lens.

### EXAMPLE 25

### Blends of Conventional Lactose and Lactose Produced in Accordance with this Invention

Particle size of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and lactose produced according to this invention combined with lactose produced according to Ser. No. 60/821,872 copending application entitled “Process for Manufacturing Lactose” filed concurrently herewith. (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA. Comparison of percentage of particles less than 1.7 μm initially and after two weeks exposure at 30 C./65% relative humidity. Blends were sized by Malvern. FIG. 6 illustrates the comparison in particle size.

### EXAMPLE 26

### Blends of Conventional Lactose and Lactose Produced in Accordance with this Invention

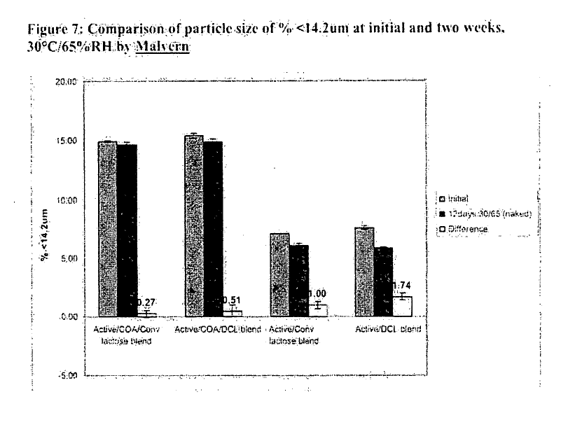

Particle size of blends of conventional fine lactose (Lot “A”, Friesland Foods Domo, Netherlands) and conventional Coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzene sulfonamide with and without cellobiose octa-acetate (“COA”) and lactose produced according to this invention combined with lactose produced according to Ser. No. 60/821,872 copending application entitled “Process for Manufacturing Lactose” filed concurrently herewith. (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA. Comparison of percentage of particles less than 4.9 μm initially and after two weeks exposure at 30 C./65% relative humidity. Blends were prepared as described in Table 5. Blends were sized by Malvern. FIG. 7 illustrates the comparison in particle size.

### EXAMPLE 27

### Blends of Conventional Lactose and Lactose Produced in Accordance with this Invention

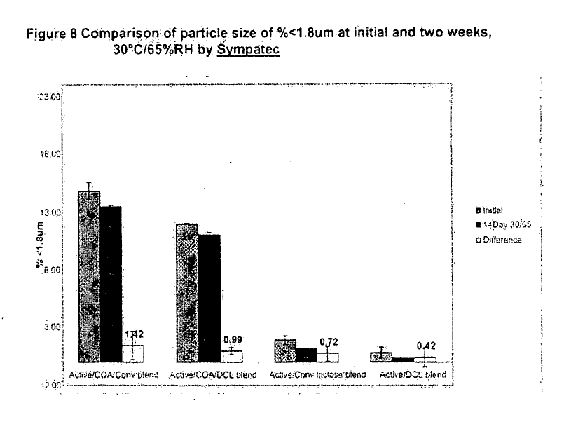

Particle size of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzene sulfonamide with and without cellobiose octa-acetate (“COA”) and lactose produced according to this invention combined with lactose produced according to Ser. No. 60/821,872 copending application entitled “Process for Manufacturing Lactose” filed concurrently herewith. (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA. Comparison of percentage of particles less than 14.2 μm initially and after two weeks exposure at 30 C./65% relative humidity. Blends were sized by Malvern. FIG. 8 illustrates the comparison in particle size.

### EXAMPLE 28

### Blends of Conventional Lactose and Lactose Produced in Accordance with this Invention

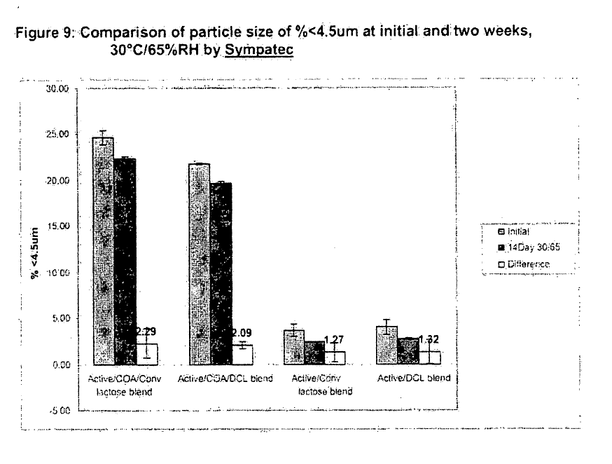

Particle size of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and lactose produced according to this invention combined with lactose produced according to Ser. No. 60/821,872 copending application entitled “Process for Manufacturing Lactose” filed concurrently herewith. (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA. Comparison of percentage of particles less than 1.8 μm initially and after weeks exposure at 30 C./65% relative humidity. Blends were sized by Sympatec. FIG. 9 illustrates the difference in particle size. For each Sympatec analysis a 2±1 g sample is transferred into the funnel of the Vibri feeder using a Kartell general purpose spatula (Fisher catalogue no. SMG-410-091M, volume approximately 1.8 cm³). The sample is then dispersed by the Vibri feeder and the Rodos disperser before entering the Sympatec HELOS laser diffraction particle sizer, model—BF or KF. Parameters: 1 bar, R4 lens.

### EXAMPLE 29

### Blends of Conventional Lactose and Lactose Produced in Accordance with this Invention

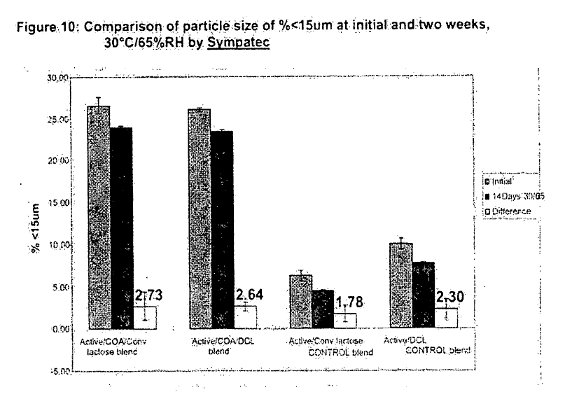

Particle size of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and lactose produced according to this invention combined with lactose produced according to Ser. No. 60/821,872 copending application entitled “Process for Manufacturing Lactose” filed concurrently herewith. (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA. Comparison of percentage of particles less than 4.5 μm initially and after two weeks exposure at 30 C./65% relative humidity. Blends were sized by Sympatec. FIG. 10 illustrates the difference in particle size. For each Sympatec analysis a 2±1 g sample is transferred into the funnel of the Vibri feeder using a Kartell general purpose spatula (Fisher catalogue no. SMG-410-091M, volume approximately 1.8 cm³). The sample is then dispersed by the Vibri feeder and the Rodos disperser before entering the Sympatec HELOS laser diffraction particle sizer, model—BF or KF. Parameters: 1 bar, R4 lens.

### EXAMPLE 30

### Blends of Conventional Lactose and Lactose Produced in Accordance with this Invention

Particle size of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and lactose produced according to this invention combined with lactose produced according to Ser. No. 60/821,872 copending application entitled “Process for Manufacturing Lactose” filed concurrently herewith. (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA. Comparison of percentage of particles less than 15 μm initially and after two weeks exposure at 30 C./65% relative humidity. Blends were sized by Sympatec. FIG. 11 illustrates the comparison in particle size. For each Sympatec analysis a 2±1 g sample is transferred into the funnel of the Vibri feeder using a Kartell general purpose spatula (Fisher catalogue no. SMG-410-091M, volume approximately 1.8 cm³). The sample is then dispersed by the Vibri feeder and the Rodos dispenser before entering the Sympatec HELOS laser diffraction particle sizer, model—BF or KF. Parameters: 1 bar, R4 lens.

### EXAMPLE 31

### Blends of Conventional Lactose and Lactose Produced in Accordance with this Invention

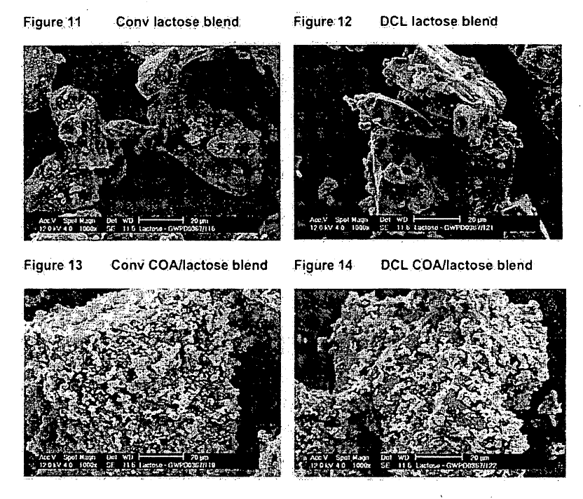

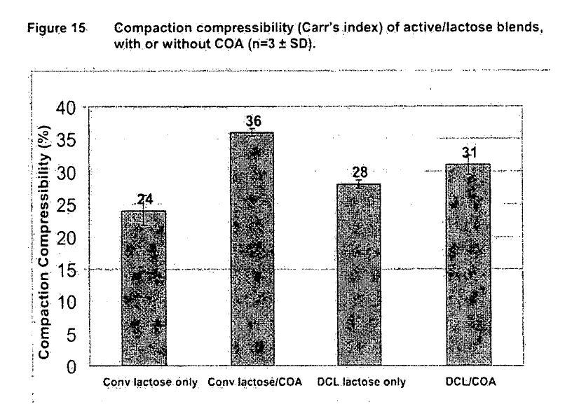

Scanning electron microscopy of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and lactose produced according to this invention combined with lactose produced according to Ser. No. 60/821,872 copending application entitled “Process for Manufacturing Lactose” filed concurrently herewith. (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA. A small amount, less than 10 mg, of each sample was finely dispersed using a small brush, onto a carbon electrodag tab stuck onto an aluminum SEM stub. These were then coated with gold using an EMSCOPE FD500 Sputter Coating Unit (Quorum Technologies, United Kingdom). The samples were then imaged on a Philips XL120 Scanning Electron Micrograph. FIGS. 12-15 illustrate various SEM photograph for these materials: FIG. 12 is an SEM of a conventional lactose blend; FIG. 13 is an SEM of a blend containing DC lactose; FIG. 14 is an SEM of a blend containing conventional lactose and COA; and FIG. 15 is an SEM of a blend containing DC lactose and COA.

### EXAMPLE 32

### Specific Surface Area Data on DCL Lactose and Conventional Lactose in Conjunction with 3-(4-{[6-({(2R)-2-hydroxy-2-[4-hydroxy-3-(hydroxymethyl)phenyl]ethyl}amino)hexyl]oxy}butyl)benzenesulfonamide

Specific surface area on blends of conventional lactose [conventional fine lactose (Lot “A”, Friesland Foods Domo, Netherlands) and conventional coarse lactose (Lot “C”, Friesland Foods Domo, Netherlands)] (“Conv”) and blends of DCL with 3-(4-{[6-({(2R)-2-hydroxy-2-[4-hydroxy-3-(hydroxymethyl)phenyl]ethyl}amino)hexyl]oxy}butyl)benzenesulfonamide (“active”) with and without micronised cellobiose octa-acetate (COA) was measured by Brunauer-Emmett-Teller (“BET”) using nitrogen as absorbate. Surface area data was obtained by measuring the quantity of gas adsorbed onto the lactose at equilibrium vapor pressure. A known quantity of nitrogen was admitted into a cell containing the sample being measured. The sample is maintained at a constant temperature, below the critical temperature of nitrogen, using liquid nitrogen. Table 7 provides the results.

TABLE 7
Specific surface area.

| | Initial | 2 wk @ | |
|---|---|---|---|
| | Surface Area | 30/65 Surface Area | % |
| Sample ID | (m² g) | (m² g) | Difference |
| conv/active blend | 0.23 (0.23-0.23) | 0.15 (0.15-0.16) | −34.78 |
| No COA | | | |
| DCL/active blend | 0.24 (0.23-0.24) | 0.19 (0.18-0.19) | −20.83 |
| No COA | | | |
| conv/active | 0.88 (0.88-0.88) | 0.74 (0.70-0.79) | −15.91 |
| blend + COA | | | |
| DCL/active | 0.86 (0.79-0.94) | 0.81 (0.79-0.83) | −5.81 |
| blend + COA | | | |
| conv lactose in | 0.22 (0.22-0.23) | 0.17 (0.15-0.19) | −22.73 |
| process sample | | | |
| DCL Lactose in | 0.25 (0.24-0.26) | 0.21 (0.21-0.22) | −16.00 |
| process sample | | | |

Values in brackets refer to range of results.

### EXAMPLE 33

### Compaction Compressibility of DCL Lactose and Conventional Lactose in Conjunction with 3-(4-{[6-({(2R)-2-hydroxy-2-[4-hydroxy-3-(hydroxymethyl)phenyl]ethyl}amino)hexyl]oxy}butyl)benzenesulfonamide

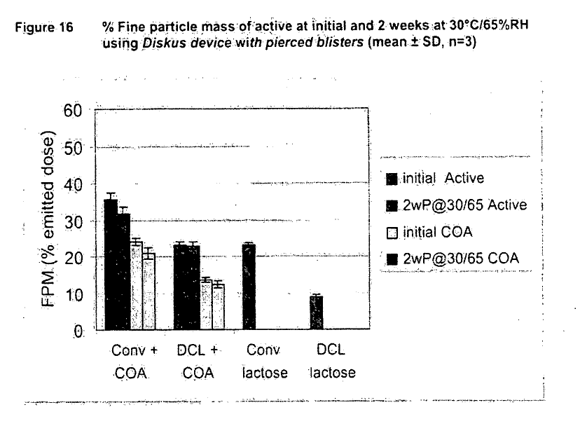

Compaction compressibility was measured on blends of conventional lactose [conventional fine lactose (Lot “A”, Friesland Foods Domo, Netherlands) and conventional coarse lactose (Lot “C”, Friesland Foods Domo, Netherlands)] (“Conv”) and blends of direct crystallized lactose (“DCL and “DC lactose”) with 3-(4-{[6-({(2R)-2-hydroxy-2-[4-hydroxy-3-(hydroxymethyl)phenyl]ethyl}amino)hexyl]oxy}butyl)benzenesulfonamide with and without micronised cellobiose octa-acetate (“COA”). The compaction compressibility was calculated from the unsettled apparent volume and final tapped volume of the blends. The unsettled apparent volume and the final tapped volume were manually recorded. The final tapped volume of the blend was recorded after the sample was subjected to 500 taps in a tap density tester Compaction compressibility=100×(Tapped Bulk Density−Initial Bulk Density)/Tapped Bulk Density. FIG. 16 illustrates the results of the compaction compressibility.

### EXAMPLE 34

### Bulk Density Summary for DCL Lactose and Conventional Lactose in Conjunction with 3-(4-{[6-({(2R)-2-hydroxy-2-[4-hydroxy-3-(hydroxymethyl)phenyl]ethyl}amino)hexyl]oxy}butyl)benzenesulfonamide

Compaction compressibility was measured on blends of conventional lactose [conventional fine lactose (Lot “A”, Friesland Foods Domo, Netherlands) and conventional coarse lactose (Lot “C”, Friesland Foods Domo, Netherlands)] (“Conv”) and direct crystallized lactose (“DCL” and “DC lactose”) with 3-(4-{[6-({(2R)-2-hydroxy-2-[4-hydroxy-3-(hydroxymethyl)phenyl]ethyl}amino)hexyl]oxy}butyl)benzenesulfonamide with and without micronised cellobiose octa-acetate (“COA”). Compaction compressibility and dynamic bulk density were calculated from the unsettled apparent volume and final tapped volume of the blends. The final tapped volume of the blend was manually recorded after the sample was subjected to 500 taps in a tap density tester. Compaction compressibility=100×(Tapped Bulk Density−Initial Bulk Density)/Tapped Bulk Density. Dynamic bulk density=(Tapped Bulk Density−Initial Bulk Density)²/Tapped Bulk Density+Initial Bulk Density. Table 8 lists the bulk density results.

TABLE 8
Bulk density summary

| | | | Compaction | |
|---|---|---|---|---|
| Sample | Initial | Tapped | Compressibility | Dynamic |
| Number | (g/ml) | (g/ml) | (%) | (g/ml) |
| active + conv lactose | 0.74 | 0.98 | 24 | 0.8 |
| Active + COA + conv | 0.65 | 1.01 | 36 | 0.8 |
| lactose | | | | |
| active + DCL | 0.58 | 0.8 | 28 | 0.6 |
| Active + DCL + COA | 0.59 | 0.85 | 31 | 0.7 |

### EXAMPLE 35

### Blends of Conventional Lactose and Lactose Produced in Accordance with this Invention

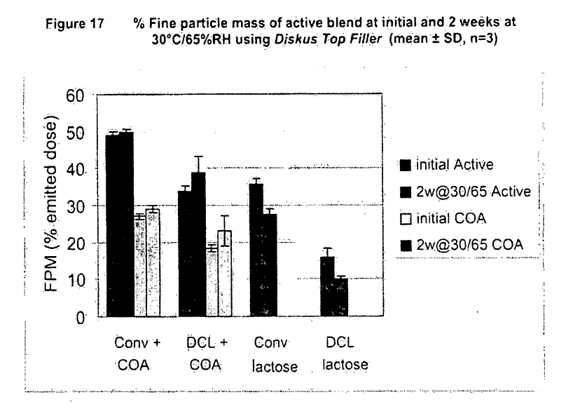

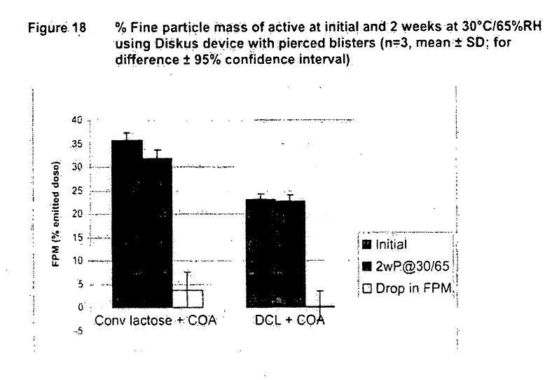

Percentage of fine particle mass of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and blends of lactose produced according to this invention combined with lactose produced Ser. No. 60/821,872 copending application entitled “Process for Manufacturing Lactose” filed concurrently herewith (“DCL” and “DCL lactose”) with 3-(4-{[6-({(2R)-2-hydroxy-2-[4-hydroxy-3-(hydroxymethyl)phenyl]ethyl}amino)hexyl]oxy}butyl)benzenesulfonamide (“drug substance”) with and without COA using Diskus® device and unpierced blisters initially and pierced blisters after two weeks exposure at 30° C./65% relative humidity (mean±SD, n=3). FIG. 17 illustrates the FPM results. All four blends were filled into 14-dose Diskus® strips using modified filling as taught in International Application No. PCT/EP00/04499. The filling equipment was set to achieve 11-16 mg, with a compaction of 10%. Blends were filled to a constant volume to ensure comparable compaction in the blister. Pierced blisters are defined as blisters which were pierced with a pin to create a hole approximately 0.14 mm². Testing was performed by reduced stage Andersen Cascade impaction at 60 L/min airflow using USP pre-separator and throat. Reduced stage Andersen Cascade impaction means that the filter was moved up the stack to sit below stage 0; anything deposited on the filter is classified as FPM. FPM of the COA and drug substance was measured by High Performance Liquid Chromatography (Dissolving solvent: 50:50 acetonitrile:water; mobile phase: 57:43 (80:20 0.01 m SDS with 0.1% acetic acid:methanol):acetonitrile; column: Zorbax C-18 50×4.6 mm 3.5 μm; flow rate: 1.5 mL/min; temperature: 40° C.; detection: UV). FPM of the lactose was quantified using High Performance Anion Exchange Chromatography (Dissolving solvent: Dissolving solvent: 50/50 Acetonitrile/water; Temperature: 40° C.; Flow rate: 1 mL/min; Mobile phase: NaOH (aqueous) 100 mM; Injection volume: 15 μL; Column: CarboPac PA-100 (4×250 mm) with guard column CarboPac PA-100 4×50 mm 10-32FTG; Detection: Pulsed Amperometric). The FPM of each component is displayed as % FPM which is calculated by dividing the deposition of that component on the filter by the total amount of that component quantified.

### EXAMPLE 36

### Blends of Conventional Lactose and Lactose Produced in Accordance with this Invention

Percentage of fine particle mass of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and blends of lactose produced according to this invention combined with lactose produced according to Ser. No. 60/821,872 copending application entitled “Process for Manufacturing Lactose” filed concurrently herewith (“DCL” and “DC lactose”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA using Diskus® Top Filler. Testing was performed initially and after two weeks exposure at 30° C./65% relative humidity. (mean±SD, n=3). FIG. 18 illustrates the FPM results. The blends were filled volumetrically into Diskus® blister sized and shaped pockets and then aerosolized through a mouthpiece with a geometry similar to the Diskus® device into the cascade impactor. Blends stored for two weeks were stored naked at 30° C./65%. Testing was performed by reduced stage Andersen Cascade impaction at 60 L/min airflow using USP pre-separator and throat. Reduced stage Andersen Cascade impaction means that the filter was moved up the stack to sit below stage 0; anything deposited on the filter is classified as FPM. FPM of the COA and drug substance was measured by HPLC (Dissolving solvent: 50:50 acetonitrile:water; mobile phase: 57:43 (80:20 0.01 m SDS with 0.1% acetic acid:methanol):acetonitrile; column: Zorbax C-18 50×4.6 mm 3.5 μm; flow rate: 1.5 mL/min; temperature: 40° C.; detection: UV). FPM of the lactose was quantified using High Performance Anion Exchange Chromatography (Dissolving solvent: Dissolving solvent: 50/50 Acetonitrile/water; Temperature: 40° C.; Flow rate: 1 mL/min; Mobile phase: NaOH (aqueous) 100 mM; Injection volume: 15 μL; Column: CarboPac PA-100 (4×250 mm) with guard column CarboPac PA-100 4×50 mm 10-32FTG; Detection: Pulsed Amperometric). The FPM of each component is displayed as % FPM which is calculated by dividing the deposition of that component on the filter by the total amount of that component quantified.

### EXAMPLE 37

### Blends of Conventional Lactose and Lactose Produced in Accordance with this Invention

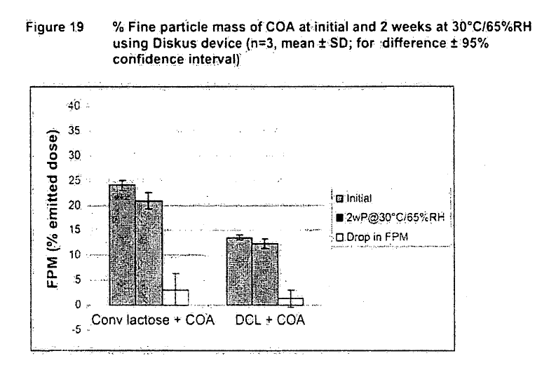

Percentage of fine particle mass of blends of conventional fine lactose (Lot “A”, Friesland Foods Domo, Netherlands) and conventional coarse lactose (Lot “C”, Friesland Foods Domo, Netherlands) (“Conv lactose”) with 3-(4-{[6-({(2R)-2-hydroxy-2-[4-hydroxy-3-(hydroxymethyl)phenyl]ethyl}amino)hexyl]oxy}butyl)benzenesulfonamide with cellobiose octa-acetate (“COA”) and blends of lactose produced according to this invention combined with lactose produced according to Ser. No. 60/821,872 copending application entitled “Process for Manufacturing Lactose” filed concurrently herewith (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA using Diskus® device. Testing was performed on unpierced blisters initially and on pierced blisters after two weeks exposure at 30° C./65% relative humidity. (mean±SD, n=3, difference in FPM±95% confidence interval). FIG. 19 illustrates the FPM results. All four blends were filled into 14-dose Diskus® strips using modified filling as taught in Example 35. The filling equipment was set to achieve 11-16 mg, with a compaction of 10%. Blends were filled to a constant volume to ensure comparable compaction in the blister. Pierced blisters are defined as blisters which were pierced with a pin to create a hole approximately 0.14 mm². Testing was performed by reduced stage Andersen Cascade impaction at 60 L/min airflow using USP pre-separator and throat. Reduced stage Andersen Cascade impaction means that the filter was moved up the stack to sit below stage 0; anything deposited on the filter is classified as FPM. FPM of the COA and drug substance was measured by HPLC (Dissolving solvent: 50:50 acetonitrile:water; mobile phase: 57:43 (80:20 0.01 m SDS with 0.1% acetic acid:methanol):acetonitrile; column: Zorbax C-18 50×4.6 mm 3.5 μm; flow rate: 1.5 mL/min; temperature: 40° C.; detection: UV). FPM of the lactose was quantified using High Performance Anion Exchange Chromatography (Dissolving solvent: Dissolving solvent: 50/50 Acetonitrile/water; Temperature: 40° C.; Flow rate: 1 mL/min; Mobile phase: NaOH (aqueous) 100 mM; Injection volume: 15 μL; Column: CarboPac PA-100 (4×250 mm) with guard column CarboPac PA-100 4×50 mm 10-32FTG; Detection: Pulsed Amperometric). The FPM of each component is displayed as % FPM which is calculated by dividing the deposition of that component on the filter by the total amount of that component quantified.

### EXAMPLE 38

### Blends of Conventional Lactose and Lactose Produced in Accordance with this Invention

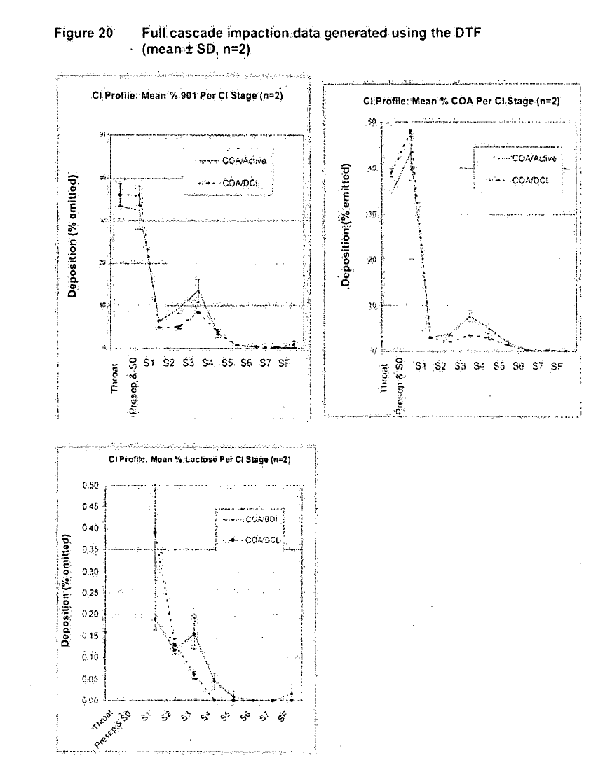

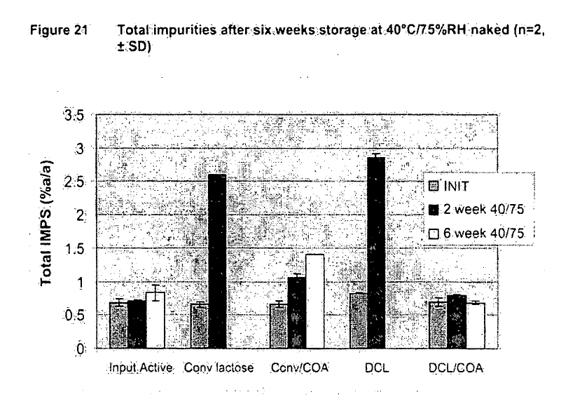

Percentage of fine particle mass of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with cellobiose octa-acetate (“COA”) and blends of lactose produced according to this invention combined with lactose produced according to Ser. No. 60/821,872 copending application entitled “Process for Manufacturing Lactose” filed concurrently herewith (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with COA using Diskus® Top Filler. Testing was performed initially and after two weeks exposure at 30° C./65% relative humidity. (mean±SD, n=3, difference in FPM±95% confidence interval). FIG. 20 illustrates the FPM results. The blends were filled volumetrically into Diskus® blister sized and shaped pockets and then aerosolized through a mouthpiece with a geometry similar to the Diskus® device into the cascade impactor. Blends stored for two weeks were stored naked at 30° C./65%. Testing was performed by reduced stage Andersen Cascade impaction at 60 L/min airflow using USP pre-separator and throat. Reduced stage Andersen Cascade impaction means that the filter was moved up the stack to sit below stage 0; anything deposited on the filter is classified as FPM. FPM of the COA and drug substance was measured by HPLC (Dissolving solvent: 50:50 acetonitrile:water; mobile phase: 57:43 (80:20 0.01 m SDS with 0.1% acetic acid:methanol):acetonitrile; column: Zorbax C-18 50×4.6 mm 3.5 μm; flow rate: 1.5 mL/min; temperature: 40° C.; detection: UV). FPM of the lactose was quantified using High Performance Anion Exchange Chromatography (Dissolving solvent: Dissolving solvent: 50/50 Acetonitrile/water; Temperature: 40° C.; Flow rate: 1 mL/min; Mobile phase: NaOH (aqueous) 100 mM; Injection volume: 15 μL; Column: CarboPac PA-100 (4×250 mm) with guard column CarboPac PA-100 4×50 mm 10-32FTG; Detection: Pulsed Amperometric). The FPM of each component is displayed as % FPM which is calculated by dividing the deposition of that component on the filter by the total amount of that component quantified.

### EXAMPLE 39

### Blends of Conventional Lactose and Lactose Produced in Accordance with this Invention

Full Andersen impaction cascade data of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with cellobiose octa-acetate (“COA”) and blends of lactose produced according to this invention combined with lactose produced according to Ser. No. 60/821,872 copending application entitled “Process for Manufacturing Lactose” filed concurrently herewith (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with COA (mean±SD, n=2) using Diskus® Top Filler. FIG. 21 illustrates the Cascade Impaction data. The letter “S” is an abbreviation for stage; for example S0 indicates stage 0. “F” is the abbreviation used for filter; the abbreviation FS stands for filter stage. The blends were filled volumetrically into Diskus® blister sized and shaped pockets and then aerosolized through a mouthpiece with a geometry similar to the Diskus® device into the cascade impactor. Testing was performed by full Andersen Cascade impaction at 60 L/min airflow using a USP pre-separator and throat. FPM was measured by HPLC (Dissolving solvent: 50:50 acetonitrile:water; mobile phase: 50:50 acetonitrile:water with 0.05% volume trifluoroacetic acid (“TFA”); column: Hypersil BDS C18, 200×4.6 mm 5 μm; flow rate: 1 mL/min; temperature: 40° C.; detection: UV for COA, fluorescence for 3-(4-{[6-({(2R)-2-hydroxy-2-[4-hydroxy-3-(hydroxymethyl)phenyl]ethyl}amino)hexyl]oxy}butyl)benzenesulfonamide).

### EXAMPLE 40

### Blends of Conventional Lactose and Lactose Produced in Accordance with this Invention

Mass median aerodynamic diameter (“MMAD”) and geometric standard deviation (“GSD”) of conventional fine lactose (Lot “A”, Friesland Foods Domo, Netherlands) combined with conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide (“active”) with and without cellobiose octa-acetate (“COA”) and lactose produced according to this invention combined with lactose produced according to Ser. No. 60/821,872 copending application entitled “Process for Manufacturing Lactose” filed concurrently herewith (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA. Table 9 lists the MMAD and GSD results. MMAD and GSD were calculated as per General Chapters: <601> AEROSOLS, NASAL SPRAYS, METERED-DOSE INHALERS, AND DRY POWDER INHALERS—METERED-DOSE INHALERS AND DRY POWDER INHALERS” United States Pharmacopeia, 2006.

TABLE 9
Mass Median Aerodynamic Diameter (MMAD)
and Geometric Standard Deviation (GSD)

| | conv lactose | conv lactose | DCL | DCL |
|---|---|---|---|---|
| | replicate 1 | replicate 2 | replicate 1 | replicate 2 |
| active MMAD | 3.1 | 3.0 | 2.8 | 2.8 |
| COA MMAD | 2.7 | 2.5 | 2.7 | 2.7 |
| active GSD | 1.5 | 1.4 | 1.6 | 1.4 |
| COA GSD | 1.5 | 1.5 | 1.8 | 1.6 |

### EXAMPLE 41

### Impurities of Crystallized Lactose Produced in Accordance with this Invention (DCL Lactose) and Conventional Lactose in Conjunction with 3-(4-{[6-({(2R)-2-hydroxy-2-[4-hydroxy-3-(hydroxymethyl)phenyl]ethyl}amino)hexyl]oxy}butyl)benzenesulfonamide

Impurities of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide (“active” 0 with and without cellobiose octa-acetate (“COA”) and blends of lactose produced according to this invention combined with lactose produced according to Ser. No. 60/821,872 copending application entitled “Process for Manufacturing Lactose” filed concurrently herewith (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA before after two weeks storage at 40° C. and 75% relative humidity. Impurities were measured using HPLC (Dissolving solvent: 10:90 ethanol:water; mobile phase: gradient from 10% 0.05% trifluoroacetic acid (“TFA”) in acetonitrile, 90% 0.05% TFA in water to 90:10 over 40 minutes; flow rate: 1 mL/min; temperature 40° C.; column Zorbax bonus RP 3.5μ 150×4.6 mm; detection: UV). Table 10 provides the impurities data.

TABLE 10
Impurities data before and after two weeks storage at 40° C./75% RH

| | | Two weeks | |
|---|---|---|---|
| | Initial mean | 40/75N | Impurities |
| Batch | impurities | impurities | increase |
| Conv + COA | 0.665 | 1.05 | 0.39 |
| DCL + COA | 0.695 | 0.80 | 0.10 |
| Conv | 0.655 | 2.60 | 1.95 |
| DCL | 0.825 | 2.85 | 2.03 |

(mean, n = 2, % area/area)

### EXAMPLE 42

### Total Impurities of Crystallized Lactose Produced in Accordance with this Invention (DCL Lactose) and Conventional Lactose in Conjunction with 3-(4-{[6-({(2R)-2-hydroxy-2-[4-hydroxy-3-(hydroxymethyl)phenyl]ethyl}amino)hexyl]oxy}butyl)benzenesulfonamide

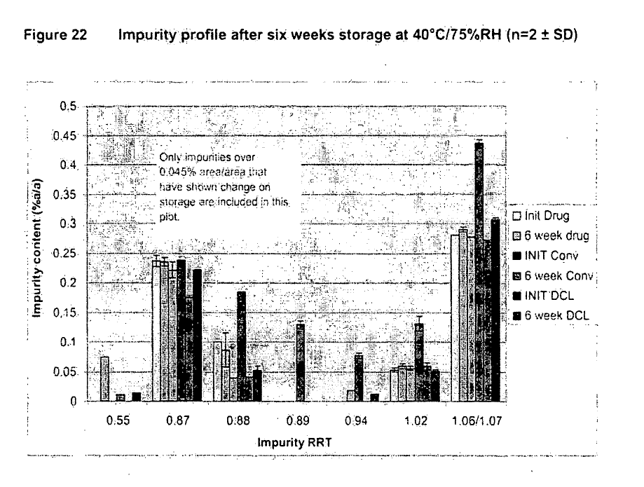

Total impurities of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and blends of lactose produced according to this invention combined with lactose produced according to Ser. No. 60/821,872 copending application entitled “Process for Manufacturing Lactose” filed concurrently herewith (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA after six weeks storage at 40° C. and 75% relative humidity. Impurities were measured using HPLC (Dissolving solvent: 10:90 ethanol-water; mobile phase: gradient from 10% 0.05% trifluoroacetic acid (“TFA”) in acetonitrile, 90% 0.05% TFA in water to 90:10 over 40 minutes; flow rate: 1 mL/min; temperature 40° C.; column Zorbax bonus RP 3.5μ 150×4.6 mm; detection: UV). FIG. 22 illustrates the impurities data.

### EXAMPLE 43

### Impurity Profile DCL Lactose and Conventional Lactose in Conjunction with 3-(4-{[6-({(2R)-2-hydroxy-2-[4-hydroxy-3-(hydroxymethyl)phenyl]ethyl}amino)hexyl]oxy}butyl)benzenesulfonamide

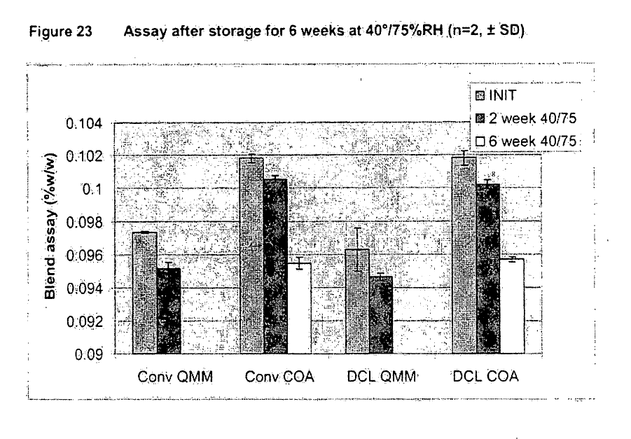

The impurity profile of blends of conventional fine lactose (Lot “A”, Friesland Foods Domo, Netherlands) and conventional coarse lactose (Lot “C”, Friesland Foods Domo, Netherlands) (“BDI”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and blends of lactose produced according to this invention combined with lactose produced according to Ser. No. 60/821,872 copending application entitled “Process for Manufacturing Lactose” filed concurrently herewith (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA after six weeks storage at 40° C. and 75% relative humidity. Impurities were measured using HPLC (Dissolving solvent: 10:90 ethanol:water; mobile phase: gradient from 10% 0.05% trifluoroacetic acid (“TFA”) in acetonitrile, 90% 0.05% TFA in water to 90:10 over 40 minutes; flow rate: 1 mL/min; temperature 40° C.; column Zorbax bonus RP 3.5μ 150×4.6 mm; detection: UV). FIG. 23 illustrates the impurities. Relative retention time is the retention time of the specific impurity in relation to the retention time of the main 3-(4-{[6-({(2R)-2-hydroxy-2-[4-hydroxy-3-(hydroxymethyl)phenyl]ethyl}amino)hexyl]oxy}butyl)benzenesulfonamide peak.

### EXAMPLE 44

### Blend Assay Data from DCL Lactose and Conventional Lactose in Conjunction with 3-(4-{[6-({(2R)-2-hydroxy-2-[4-hydroxy-3-(hydroxymethyl)phenyl]ethyl}amino)hexyl]oxy}butyl)benzenesulfonamide

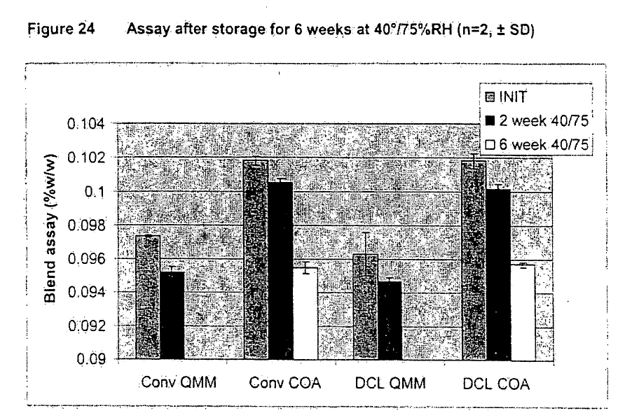

Blend assay data of conventional fine lactose (Lot “A”, Friesland Foods Domo, Netherlands) combined with conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and lactose produced according to this invention combined with lactose produced according to Ser. No. 60/821,872 copending application entitled “Process for Manufacturing Lactose” filed concurrently herewith (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA after six weeks storage at 40° C. and 75% relative humidity. Analysis was performed by HPLC (Dissolving solvent: 10:90 ethanol:water; mobile phase: gradient from 10% 0.05% trifluoroacetic acid (“TFA”) in acetonitrile, 90% 0.05% TFA in water to 90:10 over 40 minutes; flow rate: 1 mL/min; temperature 40° C.; column Zorbax bonus RP3.5μ 150×4.6 mm; detection: UV). FIG. 24 represents the assay data.

### EXAMPLE 45

### Probe Indentation Data

The change of force of probe indentation of blends of conventional fine lactose (Lot “A”, Friesland Foods Domo, Netherlands) and conventional coarse lactose (Lot “C”, Friesland Foods Domo, Netherlands) (“Conv”) with 3-(4-{[6-({(2R)-2-hydroxy-2-[4-hydroxy-3-(hydroxymethyl)phenyl]ethyl}amino)hexyl]oxy}butyl)benzenesulfonamide with and without cellobiose octa-acetate (“COA”) and lactose produced according to this invention combined with lactose produced according to Ser. No. 60/821,872 copending application entitled “Process for Manufacturing Lactose” filed concurrently herewith, (“DCL”) with 3-(4-{[6-({(2R)-2-hydroxy-2-[4-hydroxy-3-(hydroxymethyl)phenyl]ethyl}amino)hexyl]oxy}butyl)benzenesulfonamide with and without COA after 24 hours exposure at 20° C./75% relative humidity was evaluated. Probe indentation results are given in Table 11. As shown the DCL blend with COA requires the greatest force for the probe to indent the material.

TABLE 11
Change of force on storage 24 hours at 20° C. 75% RH

Change on Storage

| active batch details | Force | AreaFD |
|---|---|---|
| conv no COA | 8.47 | 17.43 |
| Conv + COA | 3.99 | 10.90 |
| DCL no COA | 14.07 | 28.20 |
| DCL + COA | 3.56 | 6.94 |

The invention has been described with respect to the embodiments described in the specification, including without limitation those set forth in the drawings and examples. It should be understood that these embodiments are merely for illustrative purposes only, and do not limit the scope of the invention as defined by the claims.

What is claimed is:

1. A process for forming crystalline lactose suitable for use in a pharmaceutical formulation, said process comprising:
subjecting a solution comprising a plurality of nanosized lactose particles to conditions sufficient to cause crystallization to occur on the nanosized lactose particles such that a plurality of lactose particles are formed therefrom having a median diameter ranging from about 4 μm to about 20 μm.

2. The process according to claim 1, further comprising the step adding a plurality of nanosized lactose particles to a second solution comprising supersaturated lactose prior to said subjecting step to form the solution comprising a plurality of nanosized lactose particles.

3. The process according to claim 1, wherein the solution comprises a base selected from the group consisting of NaOH, KOH, LiOH, and NaHCO₃.

4. The process according to claim 1, wherein the solution comprises NaOH.

5. The process according to claim 4, wherein the solution comprises 0.5 M NaOH.

6. The process according to claim 2, wherein the second solution comprises a base selected from the group consisting of NaOH, KOH, LiOH, and NaHCO₃.

7. The process according to claim 2, wherein the second solution comprises NaOH.

8. The process accordingly to claim 7, wherein the second solution comprises 0.5 M NaOH.

9. The process according to claim 8, wherein the second solution comprising 0.5 M NaOH is no more than 2% solution volume of 0.5 M NaOH.

10. The process according to claim 2, further comprising the step of adding a third solution comprising a base to the second solution prior to the addition of the plurality of nanosized lactose particles and prior to said subjecting step.

11. The process according to claim 2, further comprising the step of adding a third solution comprising NaOH to the second solution prior to the addition of the plurality of nanosized lactose particles and prior to said subjecting step.

12. The process according to claim 2, further comprising the step of adding a third solution comprising 0.5 M NaOH to the second solution prior to the addition of the plurality of nanosized lactose particles and prior to said subjecting step.

13. The process according to claim 1, wherein the solution comprises a miscible anti-solvent.

14. The process according to claim 13, wherein the miscible anti-solvent includes acetone.

15. The process according to claim 1, wherein the solution comprises a miscible anti-solvent and a base.

16. The process according to claim 15, wherein the miscible anti-solvent is selected from the group consisting of acetone, methanol, ethanol, iso-propanol, n-propanol, and tretrahydrofuran and mixtures thereof.

17. The process according to claim 2, wherein the second solution comprises a miscible anti-solvent.

18. The process according to claim 2, wherein second solution comprises a miscible anti-solvent and a base.

19. The process according to claim 18, wherein the miscible anti-solvent is selected from the group consisting of acetone, methanol, ethanol, iso-propanol, n-propanol, tretrahydrofuran, and mixtures thereof.

20. The process according to claim 18, wherein the miscible anti-solvent includes acetone.

21. The process according to claim 2, further comprising the step of adding a fourth solution comprising an anti-solvent to the second solution, prior to said step of adding a plurality of nanosized particles.

22. The solution made by the process of claim 21, wherein the second solution comprises from about 25% to 45% volume/volume anti-solvent.

23. The process according to claim 1, wherein the plurality of nanosized lactose particles have a median diameter ranging in size from about 0.2 μm to 1.0 μm.

24. The process according to claim 1, further comprising isolating the resulting crystallized lactose particles from the liquid medium.

25. The process according to claim 24, further comprising drying the resulting crystallized lactose particles.

26. The process according to claim 25, further comprising combining the resulting crystallized lactose particles with lactose particles having a median size of about 40 μm to about 100 μm to form a blend of lactose particles.

27. The process according to claim 25, further comprising combining the resulting crystallized lactose particles with at least one medicament to form a pharmaceutical formulation.

28. The process according to claim 25, further comprising combining the blend of lactose particles with at least one medicament to form a pharmaceutical formulation.

29. The process according to claim 27, wherein the pharmaceutical formulation is a dry power pharmaceutical formulation suitable for inhalation.

30. The process according to claim 27, wherein said at least one medicament is selected from the group consisting of analgesics, anginal preparations, antiinfectives, antiallergics, antihistamines, anti-inflammatories, antittussives, bronchodilators, diuretics, anticholinergics, hormones, xanthines, therapeutic proteins and peptides, salts thereof, esters thereof, solvates thereof, and combinations thereof.

31. The process according to claim 27, wherein at least one medicament includes at least one beta agonist.

32. The process according to claim 31, wherein at least one beta agonist is selected from the group consisting of salbutamol, terbutaline, salmeterol, bitolterol, formoterol, 3-(4-{[6-({(2R)-2-hydroxy-2-[4-hydroxy-3-(hydroxymethyl)phenyl]ethyl}amino)hexyl]oxy}butyl)benzenesulfonamide, 3-(3-{[7-({(2R)-2-hydroxy-2-[4-hydroxy-3-(hydroxymethyl)phenyl]ethyl}amino)heptyl]oxy}propyl)benzenesulfonamide, 4-{(1R)-2-[(6-{2-[(2,6-dichlorobenzyl)oxy]ethoxy}hexyl)amino]-1-hydroxyethyl}-2-(hydroxymethyl)phenol, 2-hydroxy-5-((1R)-1-hydroxy-2-{[2-(4-{[(2R)-2-hydroxy-2-phenylethyl]amino}phenyl)ethyl]amino}ethyl)phenylformamide, 8-hydroxy-5-{(1R)-1-hydroxy-2-[(2-{4-[(6-methoxy-1,1′-biphenyl-3-yl)amino]phenyl}ethyl)amino]ethyl}quinolin-2(1H)-one, esters thereof, solvates thereof, salts thereof and combinations thereof.

33. The process according to claim 31, wherein at least one beta agonist includes salmeterol xinafoate.

34. The process according to claim 31, wherein the at least one beta agonist includes salbutamol sulphate.

35. The process according to claim 27, wherein the at least one medicament comprise at least one anti-inflammatory steroid.

36. The process according to claim 35, wherein the at least one anti-inflammatory steroid is selected from the group consisting of mometasone, beclomethasone, budesonide, fluticasone, dexamethasone, flunisolide, triamcinolone, (6α,11β,16α,17α)-6,9-difluoro-17-{[(fluoromethyl)thio]carbonyl}-11-hydroxy-16-methyl-3-oxoandrosta-1,4-dien-17-yl 2-furoate, (6α,11β,16α,17α)-6,9-difluoro-17-{[(fluoromethyl)thio]carbonyl}-11-hydroxy-16-methyl-3-oxoandrosta-1,4-dien-17-yl 4-methyl-1,3-thiazole-5-carboxylate, esters thereof, solvates thereof, and combinations thereof.

37. The process according to claim 35, wherein the at least on anti-inflammatory steroid is comprise fluticasone propionate.

38. The process according to claim 28, where the at least one medicament comprises at least on beta agonist and at least one anti-inflammatory steroid.

39. The process according to claim 38, wherein the at least one beta agonist comprises salmeterol xinafoate and the at least one anti-inflammatory steroid comprises fluticasone propionate.

40. The process according to claim 28, wherein at least one medicament is selected from the group consisting of beclomethasone, fluticasone, flunisolide, budesonide, rofleponide, mometasone, triamcinolone, noscapine, albuterol, salmeterol, ephedrine, adrenaline, fenoterol, formoterol, isoprenaline, metaproterenol, terbutaline, tiotropium, ipatropium, phenylephrine, phenylpropanolamine, pirbuterol, reproterol, rimiterol, isotharine, tulobuterol, (−)-4-amino-3,5-dichloro-α-[[[6-[2-(2-pyridinyl)ethoxy]hexyl]methyl]benzenemethanol, esters thereof, solvates thereof, salts thereof and combinations thereof.

41. The process according to claim 28, wherein at least one medicament is selected from the group consisting of albuterol sulfate, salmeterol xinafoate, fluticasone propionate, beclomethasone dipropionate, and combinations thereof.

42. The process according to claim 28, wherein said pharmaceutical formulation further comprises at least one additional excipient.

43. The process according to claim 27, wherein said process occurs in a vessel.

44. Crystalline lactose made by the process comprising the steps of:
(A) adding a base to a supersaturated lactose solution; then
(B) adding a miscible anti-solvent to the solution; then
(C) adding a plurality of nanosized lactose particles in a water miscible organic solvent; then
(D) thereafter cooling the solution to cause crystallization; then
(E) thereafter recovering a plurality of crystallized nanosized particles having a median diameter ranging from about 4 μm to about 20 μm.

45. Crystalline lactose made by the process comprising the steps of:
(A) adding a miscible anti-solvent to a supersaturated lactose solution; then
(B) adding a plurality of nanosized lactose particles in a water miscible organic solvent to the supersaturated lactose solution; then
(C) cooling the solution to cause crystallization; then
(D) recovering a plurality of crystallized nanosized particles having a median diameter ranging from about 4 μm to about 20 μm.

46. Crystalline lactose having a median diameter (X50) ranging from about 4 μm to about 6 μm.

47. The lactose according to claim 46, wherein said lactose has logarithmic particle size distribution which is Gaussian.
